(19) United States
(12) Patent Application Publication
(10) Pub. No.: US 2024/0410005 A1
(43) Pub. Date: Dec. 12, 2024

(54) METHODS FOR PREDICTING DRUG RESPONSIVENESS IN HEART FAILURE SUBJECTS

(71) Applicant: The United States Government as represented by the Department of Veterans Affairs, Washington, DC (US)

(72) Inventors: Jacob Joseph, Washington, DC (US); Yan V. Sun, Washington, DC (US)

(21) Appl. No.: 18/699,412

(22) PCT Filed: Oct. 7, 2022
(86) PCT No.: PCT/US2022/077775

§ 371 Date: Apr. 8, 2024

Related U.S. Application Data
(60) Provisional application No. 63/253,872, filed on Oct. 8, 2021.

(51) Int. Cl. C12Q 1/6883; G01N 33/50

(52) U.S. Cl. CPC C12Q 1/6883; G01N 33/5023; C12Q 2600/106; C12Q 2600/156; G01N 2333/91017

(57) ABSTRACT

Disclosed herein are compositions and methods for identifying a subject at risk for developing heart failure (HF), heart failure with preserved ejection fraction (HFpEF), or heart failure with reduced ejection fraction (HFrEF). Described herein are also methods of treating subjects identified at risk for developing HF, HFpEF, or heart failure with reduced ejection fraction HFrEF.

### CROSS REFERENCE TO RELATED APPLICATIONS

This application claims the benefit of U.S. Provisional Application No. 63/253,872, filed Oct. 8, 2021. The content of this earlier filed application is hereby incorporated by reference herein in its entirety.

### STATEMENT REGARDING FEDERALLY FUNDED RESEARCH

This invention was made with government support under grant numbers I01-CX001737 and I01-BX004821 awarded by the Department of Veterans Affairs Office of Research and Development. The government has certain rights in the invention.

### BACKGROUND

The global epidemic of heart failure (HF) affects approximately 64 million people worldwide and 6.2 million adults in the United States (Virani S S, et al., Circulation. 2020; 141:e139-e596; and Bragazzi N L, et al. Eur J Prev Cardiol. 2021). While major advances in therapy have reduced the morbidity and mortality due to heart failure with reduced ejection fraction (HFrEF), there is significant residual risk of adverse outcomes (Teerlink J R, et al. N Engl J Med. 2021; 384:105-116). Therapeutic options are limited for heart failure with preserved ejection fraction (HFpEF), which accounts for approximately half of heart failure cases, with large scale clinical trials failing to demonstrate conclusive benefits (Solomon S D, et al. N Engl J Med. 2019; 381:1609-1620; and Shah S J, et al. Circulation. 2020; 141:1001-1026). Agents that have reduced the progression of myocardial remodeling and reduced adverse outcomes in HFrEF have not been shown to have comparable benefit in HFpEF. Absence of suitable animal models that replicate the clinical heterogeneity observed in HFpEF has hampered the understanding of pathophysiology and potential treatment targets for this highly prevalent condition (Shah S J, et al. Circulation. 2020; 141:1001-1026). There is evidence that non-cardiac comorbidities may not only increase the likelihood of adverse events in HFpEF, but may also directly affect adverse myocardial biology via proinflammatory signaling (Paulus W J and Zile M R. Circ Res. 2021; 128:1451-1467). Thus, methods for understanding the pathobiology of HFrEF and HFpEF as well as treatments for the same are needed.

### SUMMARY

Described herein are methods of identifying a subject at risk for developing heart failure (HF), heart failure with preserved ejection fraction (HFpEF), or heart failure with reduced ejection fraction (HFrEF), the methods comprising: a) obtaining a biological sample from the subject or having obtained a biological sample from the subject; b) determining the presence of one or more variants of one or more of E2F6, MITF, NFIA, METTL7A, FTO and PNMT in the biological sample; and c) identifying the subject at risk for developing HF when the one or more variants of the one or more of E2F6, MITF, NFIA, and METTL7A is present in the biological sample; identifying the subject at risk for developing HFrEF when the one or more variants of PNMT is present in the biological sample; or identifying the subject at risk for developing HFpEF, when the one or more variants of FTO is present in the biological sample.

Disclosed herein are methods of identifying heart failure in a subject that is responsive to treatment with a beta blocker, the methods comprising: a) obtaining a biological sample from the subject or having obtained a biological sample from the subject; b) determining the presence of one or more variants of PNMT in the biological sample of step a); c) contacting the biological sample in step b) with the beta blocker; d) determining a change in expression levels of PNMT in the biological sample of step c); and e) identifying the heart failure in the subject is responsive to the beta blocker when the level of expression of PNMT is different than the level of expression of PNMT in step b).

Disclosed herein are methods of treating heart failure (HF), heart failure with preserved ejection fraction (HFpEF), or heart failure with reduced ejection fraction (HFrEF) in a subject, the methods comprising: a) obtaining a biological sample from the subject or having obtained a biological sample from the subject; b) determining the presence of one or more variants of one or more of E2F6, MITF, NFIA, METTL7A, FTO and PNMT in the biological sample; c) identifying the subject having HF when the one or more variants of the one or more of E2F6, MITF, NFIA, and METTL7A is present in the biological sample; identifying the subject as having HFrEF when the one or more variants of PNMT is present in the biological sample; or identifying the subject as having HFpEF, when the one or more variants of FTO is present in the biological sample; and d) administering to the subject in step c) a therapeutically effect amount of a beta blocker, a regimen of electrocardiograms or a combination thereof, thereby treating heart failure (HF), heart failure with preserved ejection fraction (HFpEF), or heart failure with reduced ejection fraction (HFrEF) in the subject.

Disclosed herein are methods of treating a heart failure with reduced ejection fraction (HFrEF) in a subject who is responsive to a beta blocker, wherein the methods comprise the steps of: a) selecting a subject with heart failure with reduced ejection fraction (HFrEF) who is responsive to treatment with a beta blocker by: i) obtaining a biological sample from the subject or having obtained a biological sample from the subject; and ii) determining the presence of one or more variants of PNMT in the biological sample of step i); and b) based on the presence of one or more variants of PNMT, treating the subject with heart failure with reduced ejection fraction (HFrEF) with the beta blocker.

Disclosed herein are methods of determining whether a subject with heart failure (HF), heart failure with preserved ejection fraction (HFpEF), or heart failure with reduced ejection fraction (HFrEF) will respond to a therapeutic treatment, the methods comprising: a) determining the presence of one or more variants of at least one biomarker selected from the group consisting of E2F6, MITF, NFIA, METTL7A, FTO and PNMT in a sample obtained from the subject before the treatment; and b) determining a change in the expression level measured at step a) before and after contacting the sample with the therapeutic treatment; wherein detecting a difference in the biomarker expression level between the sample before and after contact with the therapeutic treatment is indicative that the subject will respond to the therapeutic treatment.

Disclosed herein are gene expression panels for assessing risk of developing heart failure (HF), heart failure with preserved ejection fraction (HFpEF), or heart failure with reduced ejection fraction (HFrEF) in a human subject, consisting of primers or probes for detecting one or more of E2F6, MITF, NFIA, METTL7A, FTO and PNMT in a sample.

### BRIEF DESCRIPTION OF THE DRAWINGS

FIG. 1 shows genome-wide significant loci association studies of HFpEF and HFrEF among non-Hispanic White veterans. Sentinel SNPs and their nearest genes are shown. *: novel HF locus; #: unique locus in the HFrEF GWAS but not in the HF meta-analysis; dashed vertical line indicates genome-wide significance threshold (P=5×10−8).

FIG. 2 shows the genetic associations between HFrEF/HFpEF risk variants and HF risk factors. Beta: beta coefficients for continuous risk factors, log (odds ratio) for binary risk factors, percent change in eGFR. CAD: coronary artery disease; AFib: atrial fibrillation; T2D: type 2 diabetes; BMI: body mass index; HDL: high-density lipoprotein cholesterol; LDL: low-density lipoprotein cholesterol; TC: total cholesterol; TG: triglycerides; SBP: systolic blood pressure; DBP: diastolic blood pressure; PP: pulse pressure; eGFR: estimated glomerular filtration rate.

FIG. 3 shows Mendelian randomization analysis of HF risk factors in relation to HFpEF and HFrEF. CAD: coronary artery disease; AFib: atrial fibrillation; T2D: type 2 diabetes; BMI: body mass index; HDL: high-density lipoprotein cholesterol; LDL: low-density lipoprotein cholesterol; TC: total cholesterol; TG: triglycerides; SBP: systolic blood pressure; DBP: diastolic blood pressure; PP: pulse pressure; eGFR: estimated glomerular filtration rate.

FIG. 4 shows sentinel SNPs significantly associated with heart failure. Note: chromosomal position is based on GCh37/hg19 reference. The sentinel SNPs were mapped to the closed refseq genes based on chromosomal base-pair position. All genetic associations were aligned to effects of the risk alleles (i.e., increased risk for unclassified HF). Ref: reference; OR: odds ratio; CI: confidence interval; GWAS: genome-wide association study. MVP—Million Veteran Program cohort (ncases=43,344) META—meta-analysis of MVP and UK Biobank cohorts).

FIG. 5 shows sentinel SNPs significantly associated with HFrEF (19,495 cases) and HFpEF (19,589 cases).

FIG. 6 shows a consort diagram detailing the phenotyping of cases (unclassified heart failure, HFrEF and HFpEF) and controls.

FIG. 7 shows genome-wide association study design of unclassified heart failure, HFrEF and HFpEF.

FIG. 8 shows quantile-quantile plot of genome-wide meta-analysis of heart failure.

FIG. 9 shows Manhattan plot of genome-wide meta-analysis of unclassified heart failure.

FIGS. 10A-T show genome-wide significant loci (n=20) associated with heart failure.

FIG. 10A-B show position on chromosome 1. FIG. 10C shows position on chromosome 2.

FIG. 10D shows position on chromosome 3. FIG. 10E shows position on chromosome 4.

FIGS. 10F-G show position on chromosome 6. FIG. 10H shows position on chromosome 7.

FIG. 10I shows position on chromosome 8. FIG. 10J shows position on chromosome 9. FIGS. 10K-L show position on chromosome 10. FIGS. 10M-N show position on chromosome 16.

FIGS. 100-R show position on chromosome 17. FIG. 10S shows position on chromosome 18.

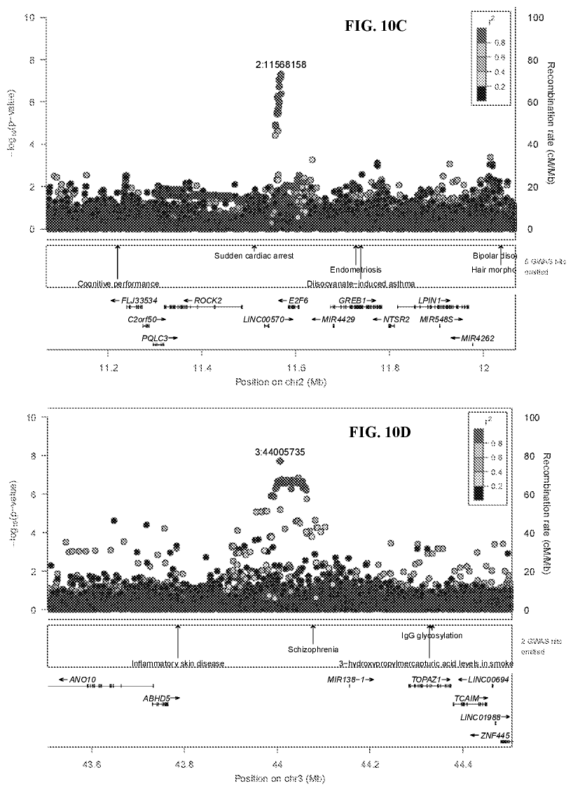

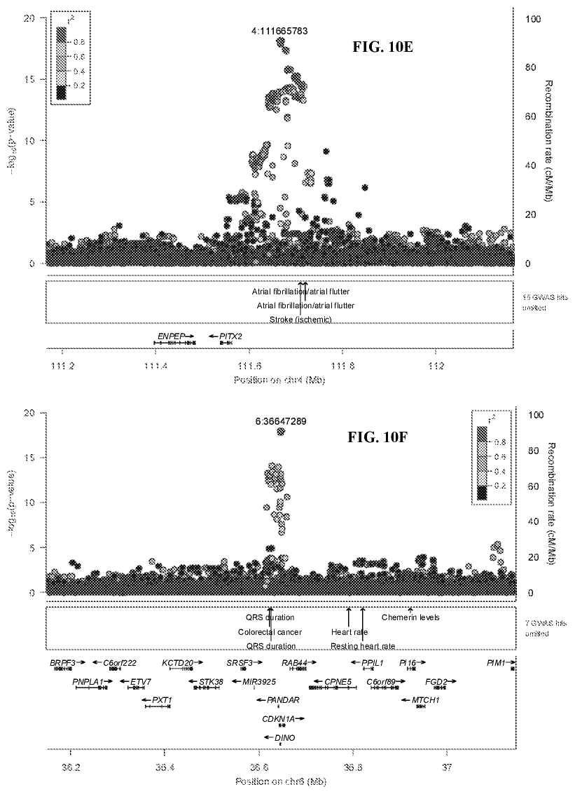

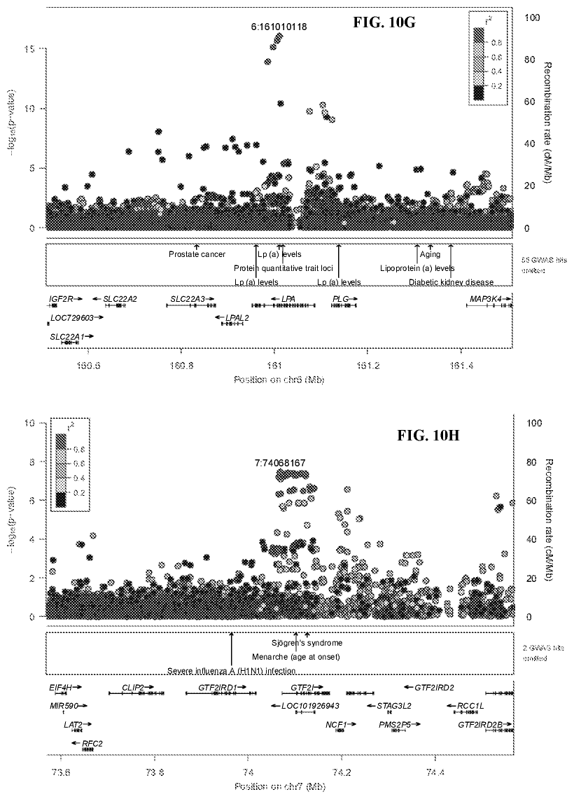

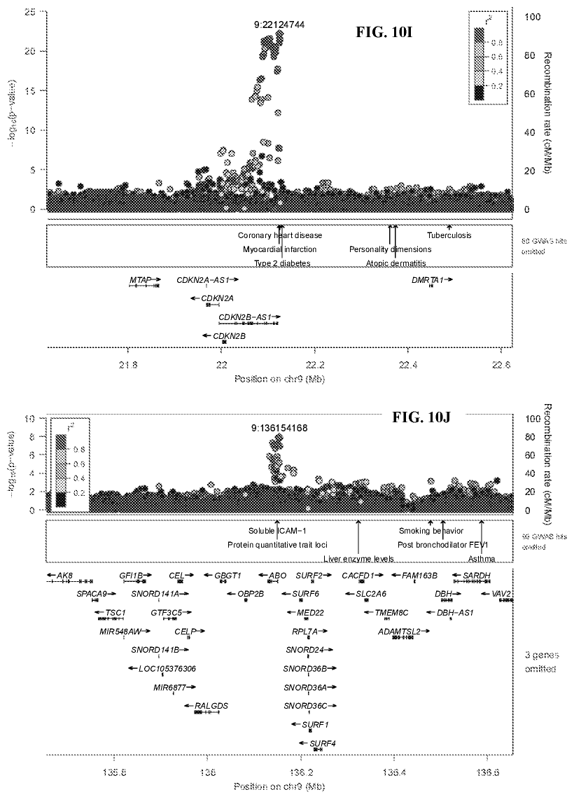

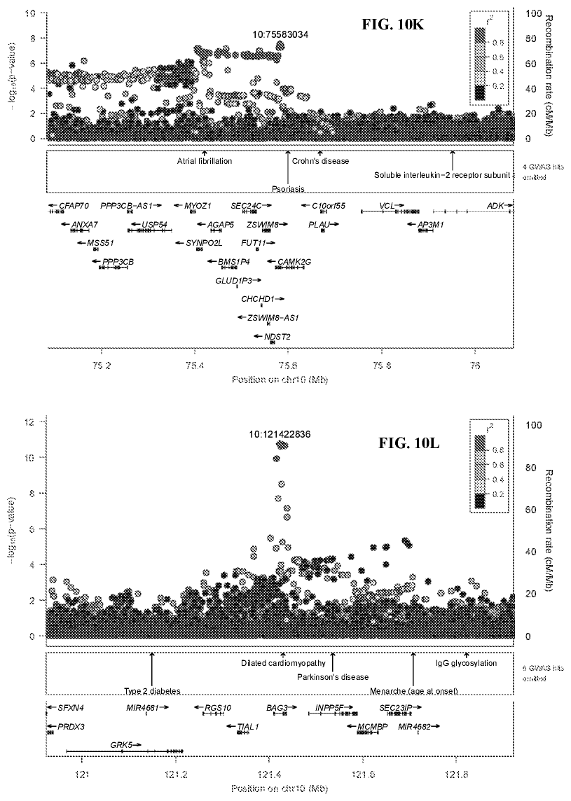

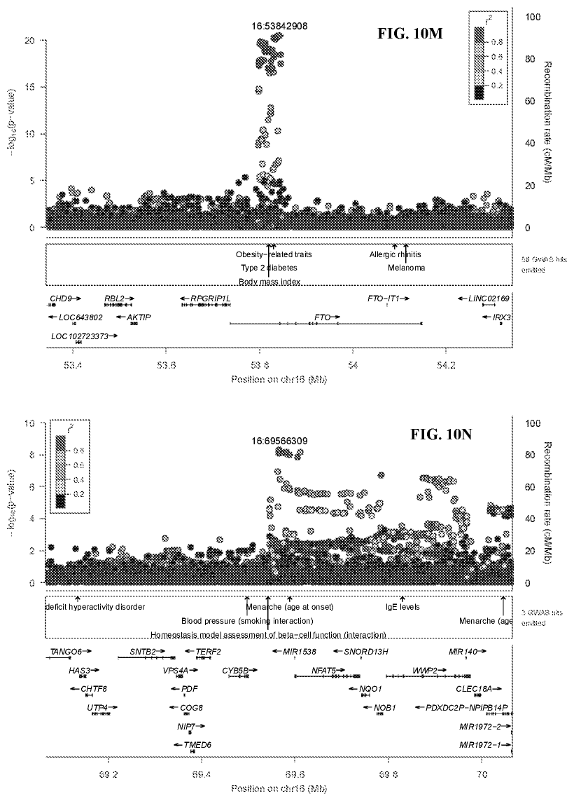

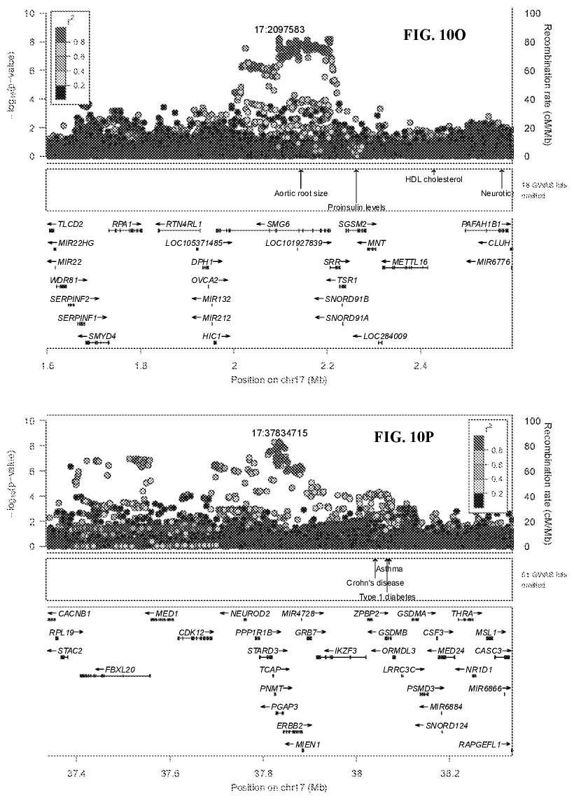

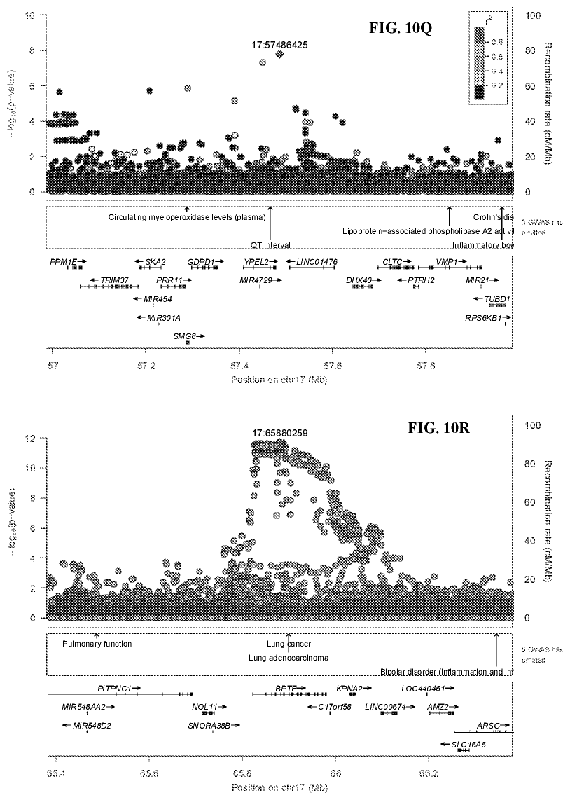

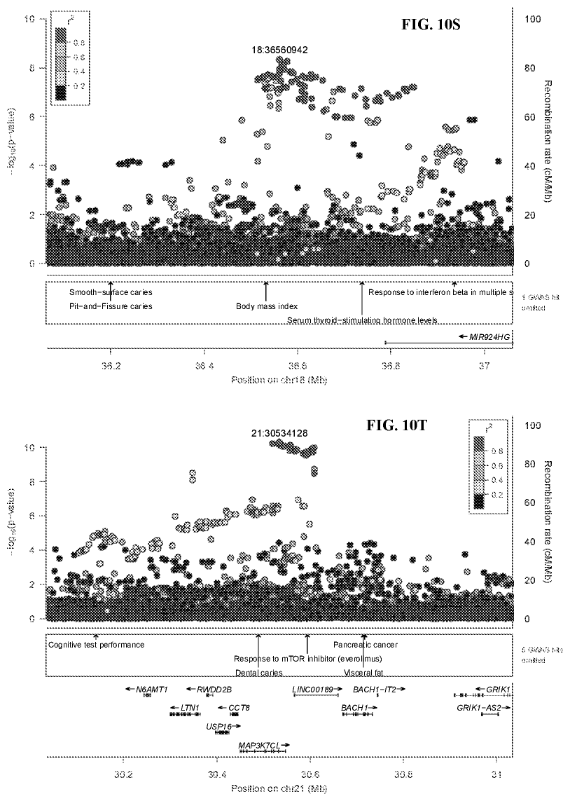

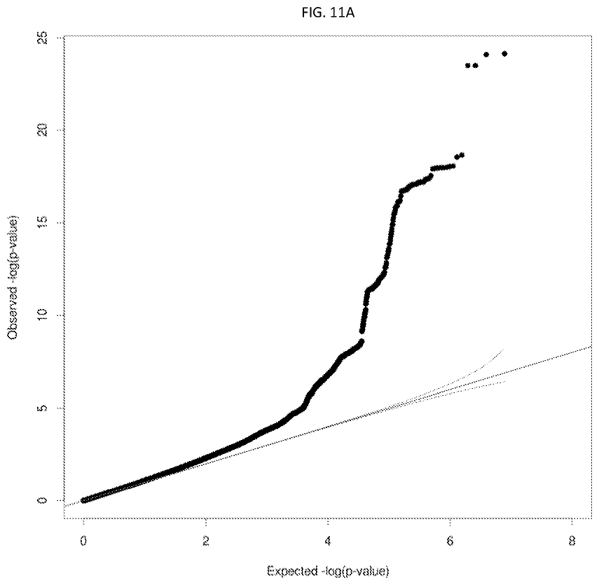

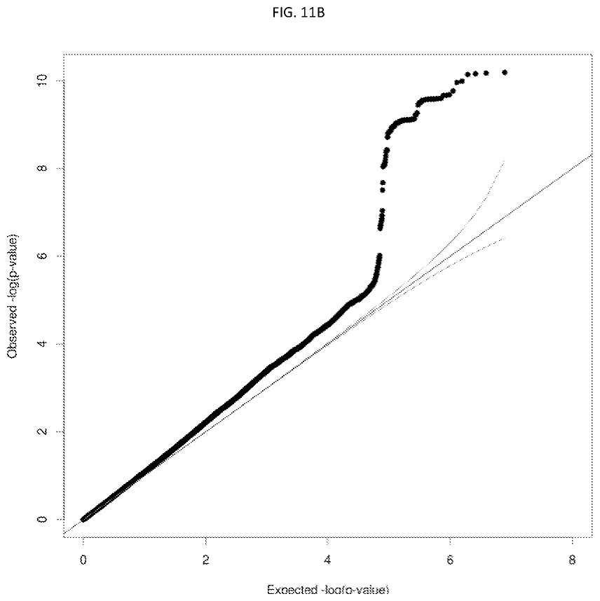

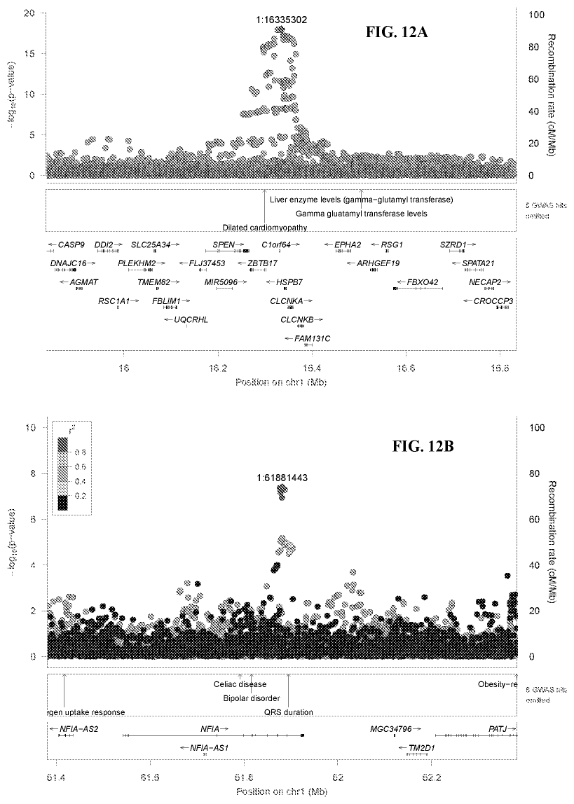

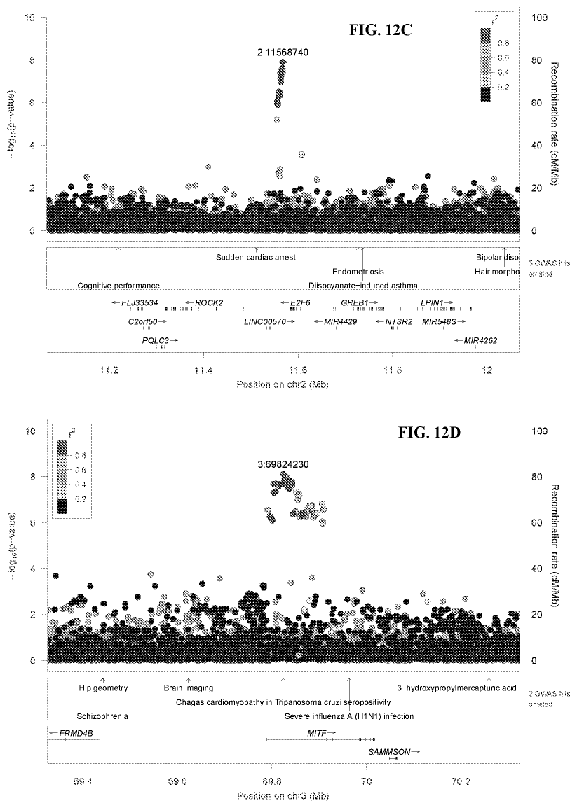

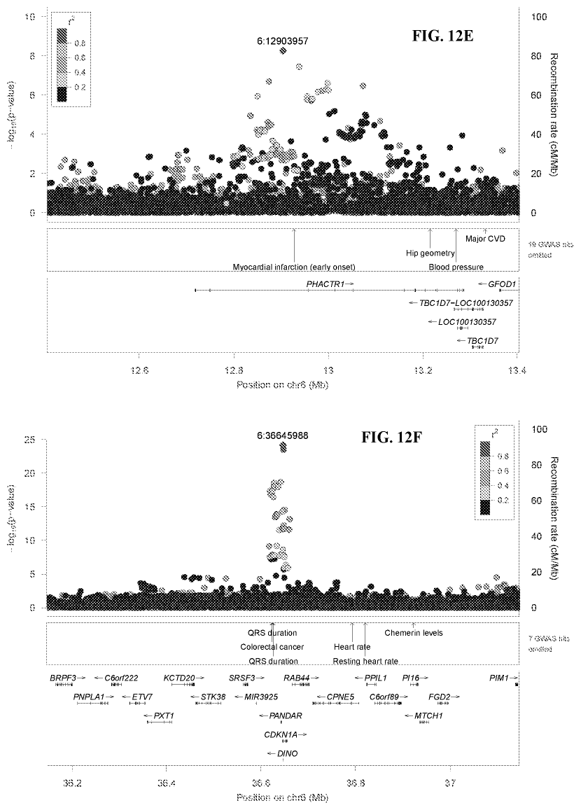

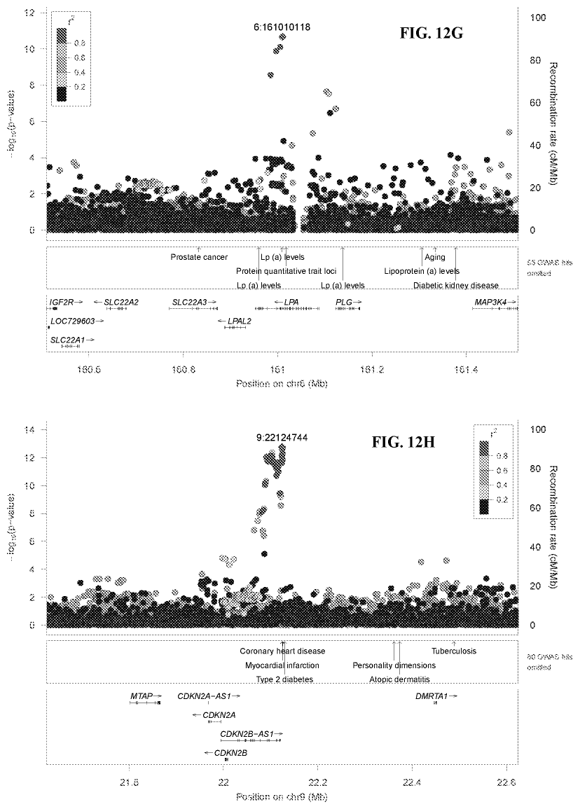

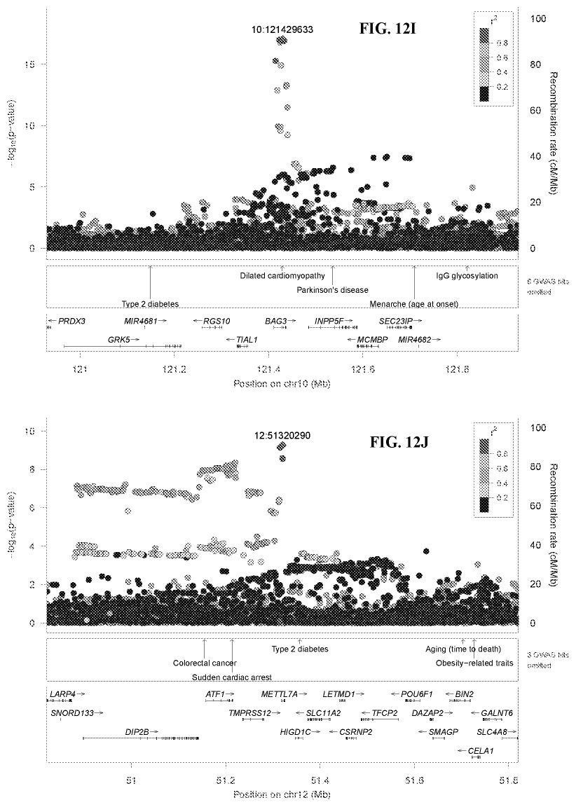

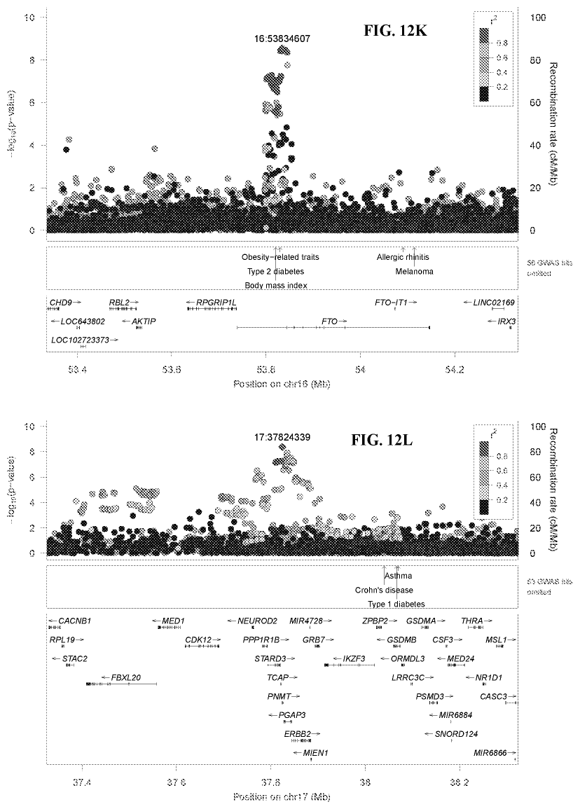

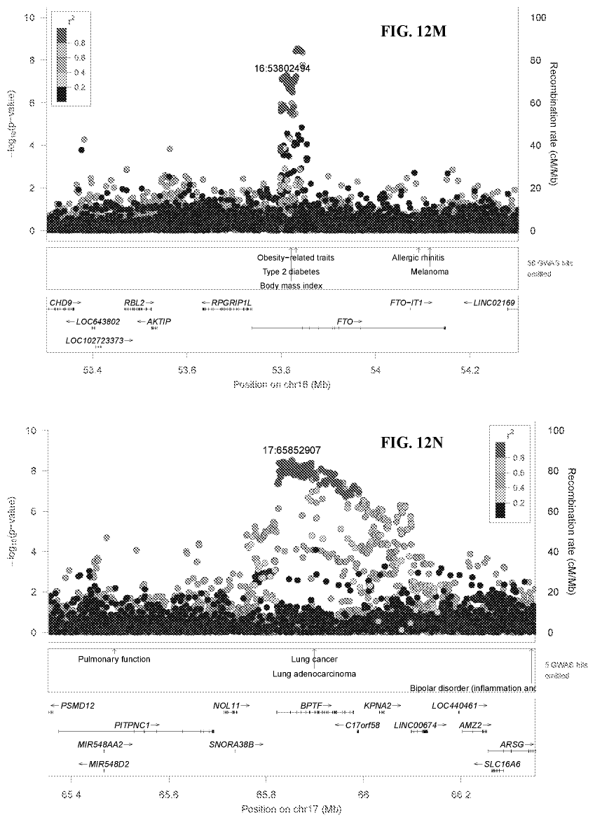

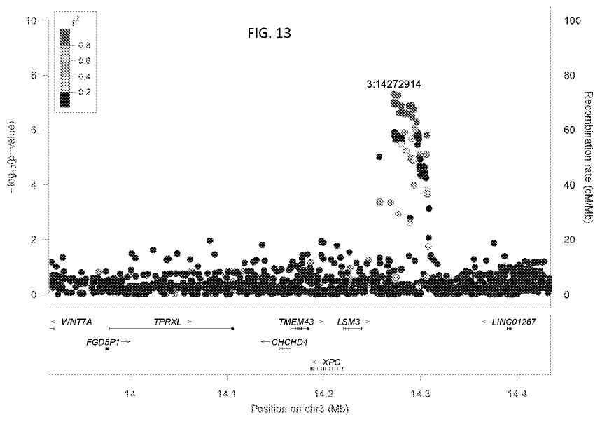

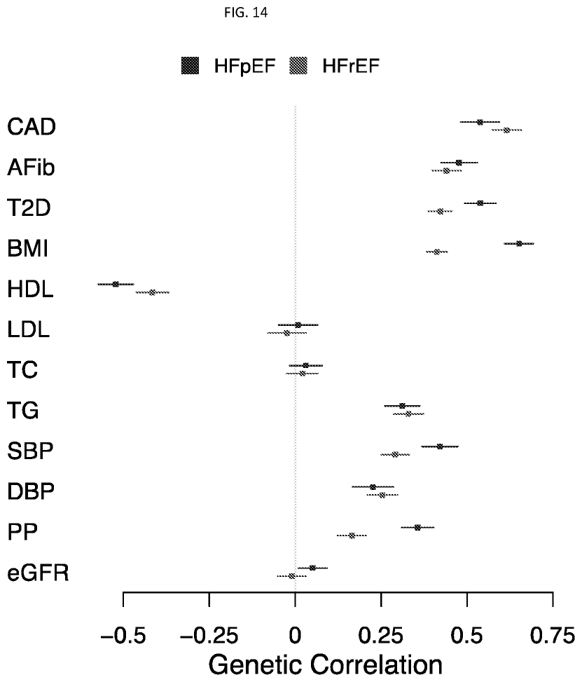

FIG. 10T shows position on chromosome 21.

FIGS. 11A-B shows quantile-quantile plots. FIG. 11A shows quantile-quantile plot of genome-wide association study of heart failure with reduced ejection fraction (HFrEF). FIG. 11B shows auantile-quantile plot of genome-wide association study of heart failure with preserved ejection fraction (HFpEF).

FIGS. 12A-N show genome-wide significant loci associated with HFrEF/HFpEF. FIG. 12A-B show position on chromosome 1. FIG. 12C shows position on chromosome 2. FIG. 12D shows position on chromosome 3. FIGS. 12E-G show position on chromosome 6. FIG. 12H shows position on chromosome 9. FIG. 12I shows position on chromosome 10. FIG. 12J shows position on chromosome 12. FIG. 12K shows position on chromosome 16. FIGS. 12L-M show position on chromosome 17. FIG. 12N genome-wide significant locus associated with HFpEF on chromosome 16.

FIG. 13 shows genetic associations of HFrEF in the TMEM43 region.

FIG. 14 shows genetic correlation between HF risk factors and HFpEF/HFrEF.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

By “treat” is meant to administer a therapeutic, such as a beta blocker, to a subject, such as a human or other mammal (for example, an animal model), that has heart failure or has an increased susceptibility for developing heart failure (including heart failure with preserved ejection fraction (HFpEF), or heart failure with reduced ejection fraction (HFrEF)), in order to prevent or delay a worsening of the effects of the disease or condition, or to partially or fully reverse the effects of the disease or condition (e.g., heart failure, including any heart failure subtypes). In some aspects, treating a subject that has heart failure or has an increased susceptibility for developing heart failure (including heart failure with preserved ejection fraction (HFpEF), or heart failure with reduced ejection fraction (HFrEF)), in order to prevent or delay a worsening of the effects of the disease or condition, or to partially or fully reverse the effects of the disease or condition (e.g., heart failure, including any heart failure subtypes) can include a regimen of electrocardiograms.

By “prevent” is meant to minimize the chance that a subject who has an increased susceptibility for developing heart failure, HFpEF or HFrEF actually develops heart failure, HFpEF or HFrEF or minimizes progression of symptoms associated with heart failure, HFpEF or HFrEF.

As used herein, the terms “administering” and “administration” refer to any method of providing a therapeutic, such as a beta blocker, to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In some aspects, the skilled person can determine an efficacious dose, an efficacious schedule, or an efficacious route of administration so as to treat a subject.

As used herein, “biological sample” refers to any sample that can be from or derived from a mammal, particularly a human patient, e.g., bodily fluids (blood, saliva, urine etc.), biopsy, tissue, and/or waste from the patient. Thus, tissue biopsies, stool, sputum, saliva, blood, plasma, serum, lymph, tears, sweat, urine, vaginal secretions, or the like can easily be screened for single nucleotide polymorphisms (SNPs), as can essentially any tissue of interest that contains the appropriate nucleic acids. These samples are typically taken, following informed consent, from a patient by standard medical laboratory methods. The sample may be in a form taken directly from the patient, or may be at least partially processed (purified) to remove at least some non-nucleic acid material.

Further, the term “sample” or “biological sample” can also mean a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of diagnosis or administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for heart failure (HF), heart failure with preserved ejection fraction (HFpEF), or heart failure with reduced ejection fraction (HFrEF), such as, for example, prior to an administering step.

As used herein, the term “comprising” can include the aspects “consisting of” and “consisting essentially of.” “Comprising” can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining the presence, quantity or an amount or a change in activity. For example, determining the presence of one or more variants of at least one biomarker selected from the group consisting of E2F6, MITF, NFIA, METTL7A, FTO and PNMT in a sample as used herein can refer to the steps that the skilled person would take to measure or detect the presence of the disclosed biomarkers in a sample. For example, determining the amount of a disclosed polypeptide, protein, gene or antibody in a sample as used herein can refer to the steps that the skilled person would take to measure or ascertain some quantifiable value of the polypeptide protein, gene or antibody in the sample. The art is familiar with the ways to measure an amount of the disclosed polypeptide, proteins, genes or antibodies in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, affection.

As used herein, the term “susceptibility” refers to the likelihood of a subject being clinically diagnosed with a disease. For example, a human subject with an increased susceptibility for heart failure (HF), heart failure with preserved ejection fraction (HFpEF), or heart failure with reduced ejection fraction (HFrEF) refer to a human subject with an increased likelihood of a subject being clinically diagnosed with heart failure (HF), heart failure with preserved ejection fraction (HFpEF), or heart failure with reduced ejection fraction (HFrEF), respectively.

As used herein, the term “gene” refers to a region of DNA encoding a functional RNA or protein. “Functional RNA” refers to an RNA molecule that is not translated into a protein. Generally, the gene symbol is indicated by using italicized styling while the protein symbol is indicated by using non-italicized styling.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

As used herein, the term “polypeptide” refers to any peptide, oligopeptide, polypeptide, gene product, expression product, or protein. A polypeptide is comprised of consecutive amino acids. The term “polypeptide” encompasses naturally occurring or synthetic molecules. As used herein, the term “amino acid sequence” refers to a list of abbreviations, letters, characters or words representing amino acid residues.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full-length polypeptides.

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

### Introduction

Disclosed herein are methods of identifying subjects at risk or an increased susceptibility for developing heart failure or a heart failure type. Also disclosed herein are method of treating subjects with heart failure or a heart failure type, as well as methods for determining whether a subject with heart failure or a heart failure type will respond to a therapeutic. Heart failure can be classified into two types, HFrEF and HFpEF. Heart failure due to reduced ejection fraction (HFrEF) is associated with ejection fraction less than 40%. Heart failure with preserved ejection fraction (HFpEF) occurs when the left ventricle contracts normally during systole, but the ventricle is stiff and does not relax normally during diastole, which impairs filling.

Genomic analyses of large cohorts are promising approaches to better understand the pathobiology of HFrEF and HFpEF (Smith N L, et al. Circ Cardiovasc Genet. 2010; 3:256-66; and Arvanitis M, et al. Nat Commun. 2020; 11:1122). A recent large meta-analysis of GWAS of unclassified HF from multiple cohorts of European ancestry have identified genomic loci associated with HF (Shah S, et al. Nat Commun. 2020; 11:163). The Million Veteran Program (MVP) is a large biobank linked to extensive national Veterans Affairs (VA) electronic health record (EHR) databases. Using algorithms developed to curate HFrEF and HFpEF phenotypes, the genetic architecture of each subtype can be extensively studied in a single large cohort in the Million Veteran Program (MVP) utilizing GWAS, Mendelian Randomization analyses, analyses of genetic associations between discovered risk variants and known HF risk factors, and analyses of genetic correlations between known HF risk factors and HFrEF and HFpEF. The results described herein demonstrate a striking difference in the genetic underpinnings of HFrEF and HFpEF and also support the urgent need for new approaches to sub-phenotype HFpEF to enable pathophysiological and therapeutic discovery.

### Methods of Identifying a Subject

Disclosed herein are methods of identifying a subject at risk for developing heart failure (HF). Also disclosed herein are methods of identifying a subject at risk for developing heart failure with preserved ejection fraction (HFpEF). Further disclosed herein are methods of identifying a subject at risk for developing heart failure with reduced ejection fraction (HFrEF). In some aspects, the methods can comprise: a) obtaining a biological sample from the subject or having obtained a biological sample from the subject; b) determining the presence of one or more variants of one or more of E2F6, MITF, NFIA, METTL7A, FTO and PNMT in the biological sample; and c) identifying the subject at risk for developing HF when the one or more variants of the one or more of E2F6, MITF, NFIA, and METTL7A is present in the biological sample; identifying the subject at risk for developing HFrEF when the one or more variants of PMNT is present in the biological sample; or identifying the subject at risk for developing HFpEF, when the one or more variants of FTO is present in the biological sample.

Disclosed herein are methods of treating heart failure (HF). Also disclosed herein are methods of treating heart failure with preserved ejection fraction (HFpEF). Further disclosed herein are methods of treating heart failure with reduced ejection fraction (HFrEF). In some aspects, the methods can comprise: a) obtaining a biological sample from the subject or having obtained a biological sample from the subject; b) determining the presence of one or more variants of one or more of E2F6, MITF, NFIA, METTL7A, FTO and PNMT in the biological sample; c) identifying the subject as having HF or at risk of developing HF when the one or more variants of the one or more of E2F6, MITF, NFIA, and METTL7A is present in the biological sample; identifying the subject as having HFrEF when the one or more variants of PNMT is present in the biological sample; or identifying the subject as having HFpEF, when the one or more variants of FTO is present in the biological sample; d) administering to the subject in step c) a therapeutically effect amount of a beta blocker, a regimen of electrocardiograms or a combination thereof.

In some aspects, the subject can have heart failure (HF), heart failure with preserved ejection fraction (HFpEF), or heart failure with reduced ejection fraction (HFrEF). In some aspects, the subject can have unclassified heart failure. In some aspects, the subject can be at risk for developing heart failure (HF), heart failure with preserved ejection fraction (HFpEF), or heart failure with reduced ejection fraction (HFrEF). In some aspects, the subject can be at risk for developing an unclassified heart failure.

In some aspects, the biological sample can be a blood sample, a DNA sample, or a nucleic acid sample. In some aspects, the DNA or nucleic acid sample can be obtained for genomics or biomarker analyses.

A DNA sample can be analyzed for known genetic variants or to discover previously unknown genetic variants in a region of interest by determining the DNA sequence in the region of interest and comparing the determined sequence to the reference sequence. In some aspects, the region of interest can be E2F6, MITF, NFIA, METTL7A, FTO and PNMT.

A genetic variant of one or more of E2F6, MITF, NFIA, METTL7A, FTO and PNMT can be a nucleic acid sequence of E2F6, MITF, NFIA, METTL7A, FTO and PNMT wherein one or more nucleotides differ from a reference DNA sequence for E2F6, MITF, NFIA, METTL7A, FTO and PNMT. For example, a genetic variant can comprise a deletion, substitution or insertion of one or more nucleotides. In some aspects, the genetic variant can have two or more single nucleotide polymorphisms (SNPs) as compared to a reference sequence.

In some aspects, the variant can be a non-genetic variant. Non-genetic variants can alter gene expression of a gene. In some aspects, non-genetic variants can alter gene expression of one or more of E2F6, MITF, NFIA, METTL7A, FTO and PNMT In some aspects, the non-genetic variant of one or more of E2F6, MITF, NFIA, METTL7A, FTO and PNMT can be one or more nucleotides which differ from a reference DNA sequence for E2F6, MITF, NFIA, METTL7A, FTO or PNMT, respectively. For example, a non-genetic variant can comprise a deletion, substitution or insertion of one or more nucleotides. In some aspects, a non-genetic variant can cause a phenotypic variation that is independent of genetic variation. In some aspects, the non-genetic variant can comprise two or more SNPs. In some aspects, the non-genetic variant can be an allele change. In some aspects, the SNP can be in the flanking region of one or more of E2F6, MITF, NFIA, METTL7A, FTO and PNMT In some aspects, the SNP can be in the intron region of FTO.

In some aspects, the non-genetic variant can be associated with unclassified heart failure. In some aspects, the non-genetic variant of E2F6 is a T to C. In some aspects, the one or more variants of E2F6 is a T to C at position 2:11568158.

In some aspects, the non-genetic variant can be associated with heart failure. In some aspects, the non-genetic variant of FTO is a G to A. In some aspects, the non-genetic variant of FTO is a G to A at position 16:53806453.

In some aspects, the non-genetic variant can be associated with HFrEF. In some aspects, the non-genetic variant of NFIA is a G to A. In some aspects, the non-genetic variant of NFIA is a G to A at position 1:61881191. In some aspects, the non-genetic variant of E2F6 is a G to A. In some aspects, the one or more variants of E2F6 is a G to A at position 2:11568740. In some aspects, the non-genetic variant of MITF is a C to G. In some aspects, the non-genetic variant of MITF is a C to G a position 3:69824230. In some aspects, the non-genetic variant of METTL7A is an A to T. In some aspects, the non-genetic variant of METTL7A is an A to T at position 12:51320290. In some aspects, the non-genetic variant of FTO is a C to T. In some aspects, the non-genetic variant of FTO is a C to T at position 16:53834607. In some aspects, the non-genetic variant of PNMT is a G to A. In some aspects, the non-genetic variant of PNMT is a G to A at position 17:37824339.

In some aspects, the non-genetic variant can be associated with HFpEF. In some aspects, the non-genetic variant of FTO is a T to C. In some aspects, the non-genetic variant of FTO is a T to C at position 16:53802494.

In some aspects, the beta blocker can be metoprolol succinate, bisoprolol, or carvedilol.

### Methods of Treating

Disclosed herein are methods of treating heart failure (HF). Disclosed herein are methods of treating heart failure with preserved ejection fraction (HFpEF). Disclosed herein are methods of treating heart failure with reduced ejection fraction (HFrEF). In some aspects, the method can comprise: a) obtaining a biological sample from the subject or having obtained a biological sample from the subject; b) determining the presence of one or more variants of one or more of E2F6, MITF, NFIA, METTL7A, FTO and PNMT in the biological sample; c) identifying the subject having HF when the one or more variants of the one or more of E2F6, MITF, NFIA, and METTL7A is present in the biological sample; identifying the subject as having HFrEF when the one or more variants of PNMT is present in the biological sample; or identifying the subject as having HFpEF, when the one or more variants of FTO is present in the biological sample; d) administering to the subject in step c) a therapeutically effect amount of a beta blocker, a regimen of electrocardiograms or a combination thereof, thereby treating heart failure (HF), heart failure with preserved ejection fraction (HFpEF), or heart failure with reduced ejection fraction (HFrEF) in the subject.

Disclosed herein are methods of treating a heart failure with reduced ejection fraction (HFrEF) patient who is responsive to a beta blocker. In some aspects, the method comprises a) selecting a subject with heart failure with reduced ejection fraction (HFrEF) who is responsive to treatment with a beta blocker by: i) obtaining a biological sample from the subject or having obtained a biological sample from the subject; ii) determining the presence of one or more variants of PNMT in the biological sample of step i); and b) based on the presence of one or more variants of PNMT, treating the heart failure patient with the beta blocker. In some aspects, the method can further comprise: iii) contacting the biological sample in step ii) with the beta blocker; iv) determining a change in expression levels of PNMT in the biological sample of step iii); and v) identifying the heart failure with reduced ejection fraction (HFrEF) in the subject as responsive to the beta blocker when the level of expression of PNMT is different than the level of expression of PNMT in step ii) after step a) ii).

Disclosed herein are methods of treating a subject with heart failure with reduced ejection fraction (HFrEF) who is responsive to a beta blocker. In some aspects, the method comprises a) selecting a subject with heart failure with reduced ejection fraction (HFrEF) who is responsive to treatment with a beta blocker by: i) obtaining a biological sample from the subject or having obtained a biological sample from the subject; ii) determining the presence of one or more variants of PNMT in the biological sample of step i); iii) contacting the biological sample in step ii) with the beta blocker; iv) determining a change in expression levels of PNMT in the biological sample of step iii); and v) identifying the heart failure with reduced ejection fraction (HFrEF) in the patient as responsive to the beta blocker when the level of expression of PNMT is different than the level of expression of PNMT in step ii); and b) based on the presence of one or more variants of PNMT, treating the heart failure patient with the beta blocker.

In some aspects, the subject can have heart failure (HF), heart failure with preserved ejection fraction (HFpEF), or heart failure with reduced ejection fraction (HFrEF). In some aspects, the subject can have unclassified heart failure.

In some aspects, the biological sample can be a blood sample, a DNA sample, or a nucleic acid sample. In some aspects, the DNA or nucleic acid sample can be obtained for genomics or biomarker analyses.

A DNA sample can be analyzed for known genetic variants or to discover previously unknown genetic variants in a region of interest by determining the DNA sequence in the region of interest and comparing the determined sequence to the reference sequence. In some aspects, the region of interest can be E2F6, MITF, NFIA, METTL7A, FTO and PNMT.

A genetic variant of one or more of E2F6, MITF, NFIA, METTL7A, FTO and PNMT can be a nucleic acid sequence of E2F6, MITF, NFIA, METTL7A, FTO and PNMT wherein one or more nucleotides differ from a reference DNA sequence for E2F6, MITF, NFIA, METTL7A, FTO and PNMT. For example, a genetic variant can comprise a deletion, substitution or insertion of one or more nucleotides. In some aspects, the genetic variant can have two or more single nucleotide polymorphisms (SNPs) as compared to a reference sequence.

In some aspects, the variant can be a non-genetic variant. Non-genetic variants can alter gene expression of a gene. In some aspects, non-genetic variants can alter gene expression of one or more of E2F6, MITF, NFIA, METTL7A, FTO and PNMT. In some aspects, the non-genetic variant of one or more of E2F6, MITF, NFIA, METTL7A, FTO and PNMT can be one or more nucleotides which differ from a reference DNA sequence for E2F6, MITF, NFIA, METTL7A, FTO or PNMT, respectively. For example, a non-genetic variant can comprise a deletion, substitution or insertion of one or more nucleotides. In some aspects, a non-genetic variant can cause a phenotypic variation that is independent of genetic variation. In some aspects, the non-genetic variant can comprise two or more SNPs. In some aspects, the non-genetic variant can be an allele change. In some aspects, the SNP can be in the flanking region of one or more of E2F6, MITF, NFIA, METTL7A, FTO and PNMT. In some aspects, the SNP can be in the intron region of FTO.

In some aspects, the non-genetic variant can be associated with unclassified heart failure. In some aspects, the non-genetic variant of E2F6 is a T to C. In some aspects, the one or more variants of E2F6 is a T to C at position 2:11568158.

In some aspects, the non-genetic variant can be associated with heart failure. In some aspects, the non-genetic variant of FTO is a G to A. In some aspects, the non-genetic variant of FTO is a G to A at position 16:53806453.

In some aspects, the non-genetic variant can be associated with HFrEF. In some aspects, the non-genetic variant of NFIA is a G to A. In some aspects, the non-genetic variant of NFIA is a G to A at position 1:61881191. In some aspects, the non-genetic variant of E2F6 is a G to A. In some aspects, the one or more variants of E2F6 is a G to A at position 2:11568740. In some aspects, the non-genetic variant of MITF is a C to G. In some aspects, the non-genetic variant of MITF is a C to G a position 3:69824230. In some aspects, the non-genetic variant of METTL7A is an A to T. In some aspects, the non-genetic variant of METTL7A is an A to T at position 12:51320290. In some aspects, the non-genetic variant of FTO is a C to T. In some aspects, the non-genetic variant of FTO is a C to T at position 16:53834607. In some aspects, the non-genetic variant of PNMT is a G to A. In some aspects, the non-genetic variant of PNMT is a G to A at position 17:37824339.

In some aspects, the non-genetic variant can be associated with HFpEF. In some aspects, the non-genetic variant of FTO is a T to C. In some aspects, the non-genetic variant of FTO is a T to C at position 16:53802494.

In some aspects, the beta blocker can be metoprolol succinate, bisoprolol, or carvedilol.

In some aspects, the step of administering to the subject in step c) can be a therapeutically effect amount of an angiotensin converting enzyme inhibitor, an angiotensin receptor blocker, an angiotensin receptor-neprilysin inhibitor, an aldosterone blocker, a hydralazine-nitrate combination or a sodium-glucose transport protein 2 inhibitor.

### Methods of Determining Responsiveness to a Treatment

Disclosed herein are methods of determining whether a subject with heart failure (HF) will respond to a therapeutic treatment. Also disclosed herein are methods of determining whether a subject with heart failure with preserved ejection fraction (HFpEF) will respond to a therapeutic treatment. Further disclosed herein are methods of determining whether a subject with heart failure with reduced ejection fraction (HFrEF) will respond to a therapeutic treatment. In some aspects, the method can comprise: a) determining the presence of one or more variants of at least one biomarker selected from the group consisting of E2F6, MITF, NFIA, METTL7A, FTO and PNMT in a sample obtained from the subject before the treatment; and b) determining a change in the expression level measured at step a) before and after contacting the sample with the therapeutic treatment; wherein detecting a difference in the biomarker expression level between the sample before and after contact with the therapeutic treatment is indicative that the subject will respond to the therapeutic treatment.

As described herein, are methods of predicting drug (or therapeutic agent) responsiveness in a sample from a subject with heart failure, heart failure with preserved ejection fraction (HFpEF), or with heart failure with reduced ejection fraction (HFrEF).

Biomarkers. As described herein, the methods described herein involve using one or more biomarkers. A biomarker can be described as a characteristic biomolecule that is differentially present in a sample taken from a subject of one phenotypic status (e.g., having a disease; or before a treatment) as compared with another phenotypic status (e.g., not having the disease; or after receiving a treatment). A biomarker can be differentially present between different phenotypic statuses if the mean or median expression level of the biomarker in the different groups is calculated to be statistically significant. Biomarkers, alone or in combination, can provide measures of relative risk or likelihood of a response to a therapeutic that a subject belongs to one phenotypic status or another. Therefore, they can be useful as markers for disease (diagnostics), therapeutic effectiveness of a drug (theranostics) and drug toxicity.

In some aspects, the biomarker can be one or more of: E2F6, MITF, NFIA, METTL7A, FTO and PNMT. In some aspects, the biomarker can be a genetic variant of one or more of E2F6, MITF, NFIA, METTL7A, FTO and PNMT. In some aspects, the biomarker can be a non-genetic variant of one or more of E2F6, MITF, NFIA, METTL7A, FTO and PNMT. In some aspects, the biomarker can be a SNP in the flanking region of one or more of E2F6, MITF, NFIA, METTL7A, FTO and PNMT. In some aspects, the SNP can be in the intron region of FTO.

In some aspects, the biomarker can be a combination of biomarkers wherein the biomarker can be one or more biomarkers selected from Table 4, one or more biomarkers selected from Table 5, one or more biomarkers selected from Table 6, one or more biomarkers selected from Table 7, one or more biomarkers selected from Table 8 or a combination thereof.

In some aspects, the one or more biomarkers disclosed herein can distinguish a subject (or a subtype of heart failure) as a responder from a non-responder to a therapeutic agent. In some aspects, the one or more biomarkers can have one or more signature patterns that can indicate that a subject (or a subtype of heart failure) will be respond to a particular treatment, therapeutic agent or therapy. In some aspects, the one or more biomarkers can have one or more signature patterns that can indicate that a subject (or a subtype of heart failure) will not respond to a particular treatment, therapeutic agent or therapy. In some aspects, the particular treatment, therapeutic agent or therapy can be a regimen of electrocardiograms. In some aspects, the particular treatment, therapeutic agent or therapy can be a beta blocker. In some aspects, the particular treatment, therapeutic agent or therapy can be an angiotensin converting enzyme inhibitor. In some aspects, the particular treatment, therapeutic agent or therapy can be an angiotensin receptor blocker. In some aspects, the particular treatment, therapeutic agent or therapy can be an angiotensin receptor-neprilysin inhibitor. In some aspects, the particular treatment, therapeutic agent or therapy can be an aldosterone blocker. In some aspects, the particular treatment, therapeutic agent or therapy can be a hydralazine-nitrate combination. In some aspects, the particular treatment, therapeutic agent or therapy can be a sodium-glucose transport protein 2 inhibitor.

In some aspects, the determining the presence of one or more variants of at least one biomarker selected from the group consisting of E2F6, MITF, NFIA, METTL7A, FTO and PNMT can be determined and compared before and after contacting a sample with a therapeutic agent, treatment or therapy. In some aspects, the presence of one or more variants of one or more biomarkers disclosed herein can be determined and compared to a reference sample.

In some aspects, the reference sample or reference gene can be NC_000002.12:c11466177-11444375 (Homo sapiens chromosome 2, GRCh38.p13 Primary Assembly; GRCh38.p13 Primary Assembly/gene=“E2F6”); NC_000003.12:69739464-69968332 (Homo sapiens chromosome 3, GRCh38.p13 Primary Assembly, MITF); C_000012.12:50925015-50932508 (Homo sapiens chromosome 12, GRCh38.p13 Primary Assembly, METTL7A); NC_000016.10:53703963-54121941 (Homo sapiens chromosome 16, GRCh38.p13 Primary Assembly; FTO); NC_000017.11:39668019-39670475 (Homo sapiens chromosome 17, GRCh38.p13 Primary Assembly, PNMT) or NC_000001.11:61077227-61462788 (Homo sapiens chromosome 1, GRCh38.p13 Primary Assembly, NFIA).

In some aspects, the non-genetic variant can be associated with unclassified heart failure. In some aspects, the non-genetic variant of E2F6 is a T to C. In some aspects, the one or more variants of E2F6 is a T to C at position 2:11568158.

In some aspects, the determination of the presence of a non-genetic variant of one or more of E2F6, MITF, NFIA, METTL7A, FTO and PNMT in a sample can indicate the subject (or a subtype of heart failure) will respond to a beta blocker. In some aspects, the determination of the presence of a non-genetic variant of one or more of E2F6, MITF, NFIA, METTL7A, FTO and PNMT in a sample compared to a reference sample can indicate the subject (or a subtype of heart failure) will respond to a beta blocker.

In some aspects, the non-genetic variant can be associated with heart failure. In some aspects, the non-genetic variant of FTO is a G to A. In some aspects, the non-genetic variant of FTO is a G to A at position 16:53806453.

In some aspects, the non-genetic variant can be associated with HFrEF. In some aspects, the non-genetic variant of NFIA is a G to A. In some aspects, the non-genetic variant of NFIA is a G to A at position 1:61881191. In some aspects, the non-genetic variant of E2F6 is a G to A. In some aspects, the one or more variants of E2F6 is a G to A at position 2:11568740. In some aspects, the non-genetic variant of MITF is a C to G. In some aspects, the non-genetic variant of MITF is a C to G a position 3:69824230. In some aspects, the non-genetic variant of METTL7A is an A to T. In some aspects, the non-genetic variant of METTL7A is an A to T at position 12:51320290. In some aspects, the non-genetic variant of FTO is a C to T. In some aspects, the non-genetic variant of FTO is a C to T at position 16:53834607. In some aspects, the non-genetic variant of PNMT is a G to A. In some aspects, the non-genetic variant of PNMT is a G to A at position 17:37824339.

In some aspects, the non-genetic variant can be associated with HFpEF. In some aspects, the non-genetic variant of FTO is a T to C. In some aspects, the non-genetic variant of FTO is a T to C at position 16:53802494.

In some aspects, the level of expression of one or more biomarkers disclosed herein can be measured and compared before and after contacting a sample with a therapeutic agent, treatment or therapy. In some aspects, the level of expression of one or more biomarkers disclosed herein can be measured and compared to a reference sample.

In some aspects, a change in levels of one or more of E2F6, MITF, NFIA, METTL7A, FTO and PNMT expression in a sample compared to a reference sample can indicate the subject (or a subtype of heart failure) will respond to a beta blocker. In some aspects, the change in levels of one or more of MITF, NFIA, METTL7A, FTO and PNMT expression in a sample will be higher compared to a reference sample can indicate the subject. In some aspects, the change in levels of one or more of MITF, NFIA, METTL7A, FTO and PNMT expression in a sample will be lower compared to a reference sample can indicate the subject.

In some aspects, no change in the levels of one or more of MITF, NFIA, METTL7A, FTO and PNMT expression in a sample compared to a reference sample can indicate the subject (or a subtype of heart failure) will not respond to a beta blocker.

In some aspects, comparison of genetic and/or non-genetic variation in samples taken at different times can reveal changes in response to therapy. For example, genetic and/or non-genetic variation or the relative frequency of a genetic and/or non-genetic variant in samples obtained during or after therapy and absent from samples obtained before therapy can reflect e.g., stage of heart failure, or other biologic responses to therapy.

In some aspects, the frequency of genetic and/or non-genetic variants, or the ratio of frequencies of different variants, can be used to predict, monitor, or evaluate response to therapy. In some aspects, the presence or the frequency of genetic and/or non-genetic variants, or the ratio of frequencies of different variants, can be used to predict the risk level or prognosis of a patient if the patient is not treated or if the patient is given one of a set of therapies.

Obtaining a tissue sample. Procedures for the extraction and collection of a sample of a subject's tissue can be done by methods known in the art. Frozen tissue specimens can also be used. In some aspects, the sample can comprise one or more cells. The sample can be whole cells or cell organelles. Cells can be collected by scraping the tissue, processing the tissue sample to release individual cells or isolating the cells from a bodily fluid. The sample can be fresh tissue, dry tissue, cultured cells or tissue. The sample can be unfixed or fixed. In some aspects, the sample can be blood. In some aspects, the sample can be a nucleic acid sample.

Measuring or determining biomarker expression levels or presence of genetic or non-genetic variants. Methods useful for determining the biomarker expression levels or the presence of genetic or non-genetic variants include carrying out amplification reactions in the region of interest such that the region of interest is present in a replicate amplification reaction is fewer than the reciprocal of the threshold frequency for a positive determination. The method thereby allows detection of the presence of genetic and non-genetic variants in a biological sample (e.g., DNA sample) even when present at very low frequency within the biological sample (e.g., DNA sample).

In some aspects, the genetic or non-genetic variant can be a single nucleotide variant that is a change from one nucleotide to a different nucleotide in the same position. In some aspects, the genetic or non-genetic variant can be an insertion or deletion that adds or removes nucleotides. In some aspects, the genetic or non-genetic variant can be a combination of multiple events including single nucleotide variants and insertions and/or deletions. In some aspects, a genetic or non-genetic variant can be composed of multiple genetic or non-genetic variants present in different regions of interest.

Amplification reactions can be performed by one-step PCR or by two-step PCR. In some aspects, amplification reactions are performed using one or more primer pairs flanking the region of interest which integrate sample and/or amplification reaction replicate specific identifier sequences into the products of amplification. Identifier sequences can be defined as any series of DNA bases that is sufficiently different from another series of DNA bases such that when read along with an attached targeted region of interest, the identifier can be used to identify from which sample and/or amplification reaction replicate the targeted sequence originated.

Expression levels of one or more of the genes described herein or the presence of one or more variants of one or more of the genes described herein can be also be determined indirectly by determining the mRNA expression for the one or more genes in a tissue sample. RNA expression methods include but are not limited to extraction of cellular mRNA and Northern blotting using labeled probes that hybridize to transcripts encoding all or part of the gene, amplification of mRNA using gene-specific primers, polymerase chain reaction (PCR), and reverse transcriptase-polymerase chain reaction (RT-PCR), followed by quantitative detection of the gene product by a variety of methods; extraction of RNA from cells, followed by labeling, and then used to probe cDNA or olignonucleotides encoding the gene, in situ hybridization; and detection of a reporter gene.

In some aspects, the cut-off for determining the presence of a non-genetic variant in sequencing results for an amplification reaction can be ≥1.5 times compared to a control.

As used herein, the term “reference,” “reference expression,” “reference sample,” “reference value,” “control,” “control sample” and the like, when used in the context of a sample or expression level of one or more genes or proteins refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from heart failure). The reference value can be a predetermined standard value or a range of predetermined standard values, representing no illness, or a predetermined type or severity of illness.

Reference expression can be the level of the one or more genes described herein in a reference sample from a subject, or a pool of subjects, not suffering from heart failure or from a predetermined severity or type of heart failure. In some aspects, the reference value is the level of one or more genes disclosed herein in the tissue of a subject, or subjects, wherein the subject or subjects is not suffering from heart failure. In some aspects, the reference sample can be the known genetic sequence of any one of E2F6, MITF, NFIA, METTL7A, FTO and PNMT.

Reference expression can be the level of the one or more genes or biomarkers described herein in a reference sample from a subject, or a pool of subjects, not suffering from heart failure or with a known response (or lack thereof) to a particular treatment. In some aspects, the reference value can be the level of one or more genes disclosed herein in the r biological sample of a subject, or subjects, wherein the subject or subjects known to be a responder to a particular therapeutic agent or is known to be no be responsive to a particular therapeutic agent. In some aspects, the reference value can be the level of one or more genes disclosed herein in the biological sample of the same subject before or after administration of or exposure to a particular therapeutic agent. In some aspects, the reference value can be taken a different time point than to which it is being compared.

As used herein, a “reference value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual before administration of or exposure to a particular therapeutic agent, but at an earlier point in time, or a value obtained from a sample from cancer patient other than the individual being tested, or a “normal” individual, that is an individual not diagnosed with cancer. The reference value can be based on a large number of samples, such as from cancer patients or normal individuals or based on a pool of samples including or excluding the sample to be tested. The reference value can also be based on a sample from cancer patient other than the individual being tested, or a “normal” individual that is an individual not diagnosed with cancer that has not or has been administered or exposed to a particular therapeutic agent.

The reference level used for comparison with the measured level for any of the biomarkers disclosed herein can vary, depending the method begin practiced, as will be understood by one of ordinary skill in the art. For methods for determining the likelihood a cancer, a subject or a sample will be responsive to a particular type of therapeutic agent or treatment, the “reference level” is typically a predetermined reference level, such as an average of levels obtained from a population that has either been exposed or has not been exposed to particular type of therapeutic agent or treatment, but in some instances, the reference level can be a mean or median level from a group of individuals that are responders or non-responders. In some instances, the predetermined reference level can be derived from (e.g., is the mean or median of) levels obtained from an age-matched population.

Age-matched populations (from which reference values may be obtained) can be populations that are the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

The method of comparing a measured value and a reference value or a measured value before and after contact with a therapeutic agent can be carried out in any convenient manner appropriate to the type of measured value or any of the other biomarkers disclosed herein. For example, ‘measuring’ can be performed using quantitative or qualitative measurement techniques, and the mode of comparing a measured value and a reference value can vary depending on the measurement technology employed. For example, the measured values used in the methods described herein can be quantitative values (e.g., quantitative measurements of concentration, such as nanograms of the biomarker per milliliter of sample, or absolute amount). As with qualitative measurements, the comparison can be made by inspecting the numerical data, by inspecting representations of the data (e.g., inspecting graphical representations such as bar or line graphs).

### Gene Expression Panel

Disclosed herein are gene expression panels and arrays for assessing risk of developing heart failure (HF), heart failure with preserved ejection fraction (HFpEF), or heart failure with reduced ejection fraction (HFrEF) in a human subject comprising one or more primers or probes capable of detecting one or more genes or variants disclosed herein. The disclosed gene expression panels or arrays can comprise any of the genes or variants disclosed herein. For example, the disclosed gene expression panels or arrays can be used to detect one or more of E2F6, MITF, NFIA, METTL7A, FTO and PNMT or variants thereof. In some aspects, the gene expression panels or arrays can comprise E2F6, MITF, NFIA, METTL7A, FTO and PNMT or variants thereof. In some aspects, the gene expression panels or arrays can comprise primers or probes capable of detecting one or more of E2F6, MITF, NFIA, METTL7A, FTO and PNMT or a variant thereof. In some aspects, the gene expression panels or arrays can exclude one or more of the biomarkers or variants disclosed herein.

In some aspects, the biological sample can be a blood sample, a DNA sample, or a nucleic acid sample. In some aspects, the DNA or nucleic acid sample can be obtained for genomics or biomarker analyses.

The gene expression panels or arrays disclosed herein can consist of primers or probes capable of detecting or amplifying any number of the genes disclosed herein. The gene expression panels or arrays disclosed herein can further comprise primers or probes capable of detecting or amplifying any number of genes not disclosed herein. For example, the primers or probes can detect or amplify between 1 and 5, 5 and 10, 10 and 100, or more, or any variation in between.

The gene expression panels or arrays disclosed herein can be used as a standalone method for assessing risk of developing heart failure or a type of heart failure in a subject or in combination with one or more other gene expression panels or arrays not disclosed herein.

They can be used along with one or more diagnostic test. In some aspects, the gene expression panels or arrays can further comprise a second diagnostic test. The gene expression panels or arrays disclosed herein can also be used in methods to generate a specific profile. The profile can be provided in the form of a heatmap or boxplot.

The profile of the gene expression levels can be used to compute a statistically significant value based on differential expression of the one or more genes disclosed herein, wherein the computed value correlates to a diagnosis for a subtype of heart failure. The variance in the obtained profile of expression levels of the said selected genes or gene expression products can be either upregulated or downregulated in subjects with an increased susceptibility compared to a reference subject or control. The Examples section provides additional detail. For instance, when the expression level of one or more of E2F6, MITF, NFIA, METTL7A, FTO and PNMT are upregulated, the expression level indicates an increased risk of developing heart failure or a type of heart failure. When the expression level of one or more of E2F6, MITF, NFIA, METTL7A, FTO and PNMT, for instance, is downregulated, this can also indicate an increased risk of developing heart failure or a type of heart failure. As described herein, one of ordinary skill in the art can use a combination of any of genes disclosed herein to form a profile that can then be used to assess risk of developing one or more of E2F6, MITF, NFIA, METTL7A, FTO and PNMT, or to determine (and diagnose) whether a subject has one or more of E2F6, MITF, NFIA, METTL7A, FTO and PNMT.

Disclosed herein are methods of diagnosing one or more of E2F6, MITF, NFIA, METTL7A, FTO and PNMT using the gene expression panel or array described herein. In an aspect, the method further comprises performing an electrocardiogram.

In some aspects, the gene expression panel or array disclosed herein can be used to determine or assess the risk of developing heart failure or a type of heart failure in a subject, wherein the expression level for one or more of E2F6, MITF, NFIA, METTL7A, FTO and PNMT, in the sample from the subject is compared to a reference expression level for one or more of E2F6, MITF, NFIA, METTL7A, FTO and PNMT. In some aspects, the gene expression panel or array disclosed herein can be used to determine or assess the risk of developing heart failure or a type of heart failure in a subject, wherein a ratio (or percent change) of the expression level of one or more of E2F6, MITF, NFIA, METTL7A, FTO and PNMT of the subject's sample to the reference expression level of one or more of E2F6, MITF, NFIA, METTL7A, FTO and PNMT indicates a change in expression level of one or more of E2F6, MITF, NFIA, METTL7A, FTO and PNMT in the sample. In an aspect, the ratio (or percent change) of the subject's sample expression level of one or, two or more, three or more, four or more, five or more, or six of E2F6, MITF, NFIA, METTL7A, FTO and PNMT to the reference expression level of two or more, three or more, four or more, five or more of E2F6, MITF, NFIA, METTL7A, FTO and PNMT indicates a higher or lower expression level of two or more, three or more, four or more, five or more, of E2F6, MITF, NFIA, METTL7A, FTO and PNMT in the sample, indicating that the subject has an increased susceptibility to heart failure or a type of heart failure. Suitable statistical and other analysis can be carried out to confirm a change (e.g., an increase or a higher level of expression, or a decrease or a lower level of expression) in one or more of E2F6, MITF, NFIA, METTL7A, FTO and PNMT when compared with a reference sample.

The gene expression panel or array can consist of primers or probes capable of detecting, amplifying or otherwise measuring the presence or expression of one or more genes disclosed herein. For example, specific primers that can be used in the methods disclosed herein include, but are not limited to the primers suitable for use in the standard exon array from the Affymetrix website listed at: affYmetrix.com. In some aspects, the gene expression panel or array disclosed herein can be used to determine or assess the risk of developing heart failure or heart failure type in a subject, wherein E2F6, MITF, NFIA, METTL7A, FTO and PNMT RNA expression levels are detected in the sample.

In some aspects, a diagnostics kit is disclosed comprising one or more probes or primers capable of detecting, amplifying or measuring the presence or expression of one or more genes or variants disclosed herein.

Disclosed herein, are solid supports comprising one or more primers, probes, polypeptides, or antibodies capable of hybridizing or binding to one or more of the genes disclosed herein. Solid supports are solid state substrates or supports that molecules, such as analytes and analyte binding molecules, can be associated. Analytes (e.g., calcifying nanoparticles and proteins) can be associated with solid supports directly or indirectly. For example, analytes can be directly immobilized on solid supports. Analyte capture agents (e.g., capture compounds) can also be immobilized on solid supports.

As mentioned above, one of ordinary skill in the art can determine the expression level of one or more genes (or proteins) disclosed herein any number of ways. To detect or quantify the level of RNA products of the biomarkers within a sample, arrays, such as microarrays, RT-PCR (including quantitative RT-PCR), nuclease protection assays and Northern blot analyses can be used. Accordingly, in some aspects, the biomarker expression levels can be determined using arrays, microarrays, RT-PCR, quantitative RT-PCR, nuclease protection assays or Northern blot analyses.

An array is a form of solid support. An array detector is also a form of solid support to which multiple different capture compounds or detection compounds have been coupled in an array, grid, or other organized pattern.

Solid-state substrates for use in solid supports can include, for instance, any solid material to which molecules can be coupled. Examples of such materials include acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, poly lactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or any combination thereof. Solid-state substrates and solid supports can be porous or non-porous. An example of a solid-state substrate is a microtiter dish (e.g., a standard 96-well type). A multiwell glass slide can also be used. For example, such as one containing one array per well can be used, allowing for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array can generally have one type of component (that is, all the components at that location are the same). Each location can have multiple copies of the component. The spatial separation of different components in the array allows separate detection and identification of the polynucleotides or polypeptides disclosed herein.

It is not required that a given array be a single unit or structure. The set of compounds can be distributed over any number of solid supports. For example, each compound can be immobilized in a separate reaction tube or container, or on separate beads or microparticles. Different aspects of the disclosed method and use of the gene expression panel or array or diagnostic device can be performed with different components (e.g., different compounds specific for different proteins) immobilized on a solid support.

Some solid supports can have capture compounds, such as antibodies, attached to a solid-state substrate. Such capture compounds can be specific for calcifying nanoparticles or a protein on calcifying nanoparticles. Captured calcified nanoparticles or proteins can then be detected by binding of a second detection compound, such as an antibody. The detection compound can be specific for the same or a different protein on the calcifying nanoparticle.

Methods for immobilizing nucleic acids, peptides or antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidinbiotin, photocrosslinkable agents, epoxides, maleimides and N-[y-Maleimidobutyryloxy] succinimide ester (GMBS), and a heterobifunctional crosslinker. Antibodies can be attached to a substrate by chemically cross-linking a free amino group on the antibody to reactive side groups present within the solid-state substrate. Antibodies can be, for example, chemically cross-linked to a substrate that contains free amino, carboxyl, or sulfur groups using glutaraldehyde, carbodiimides, or GMBS, respectively, as cross-linker agents. In this method, aqueous solutions containing free antibodies can be incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For crosslinking with GMBS, glass substrates can be chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides can be activated by immersing in a 0.5 mg/ml solution of GMBS in 1% dimethylformamide, 99% ethanol for 1 hour at room temperature. Antibodies or proteins can be added directly to the activated substrate, which can be blocked with solutions containing agents such as 2% bovine serum albumin, and air-dried. Other standard immobilization chemistries are known by those of ordinary skill in the art.

Each of the components (e.g., compounds) immobilized on the solid support can be located in a different predefined region of the solid support. Each of the different predefined regions can be physically separated from each other. The distance between the different predefined regions of the solid support can be either fixed or variable. For example, in an array, each of the components can be arranged at fixed distances from each other, while components associated with beads will not be in a fixed spatial relationship. The use of multiple solid support units (e.g., multiple beads) can result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

In addition, the genes and variants described herein can also be used as markers (i.e., biomarkers) for susceptibility to or presence or progression of heart failure or type of heart failure. The methods and assays described herein can be performed over time, and the change in the level of the markers assessed. For example, the assays can be performed every 24-72 hours for a period of 6 months to 1 year, and thereafter carried out as needed. Assays can also be completed prior to, during, or after a treatment protocol. Together, the genes disclosed herein can be used to profile an individual's risk or progression of colon cancer. As used within this context, the terms “differentially expressed” or “differential expression” refers to difference in the level of expression of the biomarkers disclosed herein that can be assayed by measuring the level of expression of the products (e.g., RNA or gene product) of the biomarkers, such as the difference in level of messenger RNA transcript or a portion thereof expressed or of proteins expressed of the biomarkers. In some aspects, this difference is significantly different.

To improve sensitivity, more than one gene disclosed herein can be assayed within a given sample. Binding agents specific for different proteins, antibodies, nucleic acids provided herein can be combined within a single assay. Further, multiple primers or probes can be used concurrently. To assist with such assays, specific biomarkers can assist in the specificity of such tests.

Levels of expression can be measured at the transcriptional and/or translational levels. At the translational level, expression of any of the genes described herein can be measured using immunoassays including immunohistochemical staining, western blotting, ELISA and the like with an antibody that selectively binds to the corresponding gene or a fragment thereof. Detection of the protein using protein-specific antibodies in immunoassays is known in the art. At the transcriptional level, mRNA can be detected by, for example, amplification (e.g., PCR, LCR), or hybridization assays (e.g., northern hybridization, RNAse protection, or dot blotting). The level of protein or mRNA can be detected, for example, by using directly or indirectly labeled detection agents (e.g., fluorescently or radioactively labeled nucleic acids, radioactively or enzymatically labeled antibodies). Changes (e.g., increase or decrease) in the transcriptional levels can also be measured using promoter-reporter gene fusion constructs. For example, the promoter region of a gene encoding any of the genes disclosed herein can be fused (i.e., operably linked) to the coding sequence of a polypeptide that produces a detectable signal. Reporter constructs are well known in the art. Examples of reporter sequences include fluorescent proteins (e.g., green, red, yellow), phosphorescent proteins (e.g., luciferase), antibiotic resistance proteins (e.g., beta lactamase), enzymes (e.g., alkaline phosphatase).

### Kits

In some aspects, kits are provided for measuring the binding of a primer or probe to one or more biomarkers disclosed herein. The kits can comprise materials and reagents that can be used for measuring the expression level of the antibodies to one or more biomarkers. Examples of suitable kits include RT-PCR or microarray. These kits can include the reagents needed to carry out the measurements of the gene or variant expression levels. Alternatively, the kits can further comprise additional materials and reagents. For example, the kits can comprise materials and reagents required to measure gene or variant expression levels of any number of biomarkers up to 1, 2, 3, 4, 5, 10, or more biomarkers that are not biomarkers disclosed herein.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of condition disorder or disease.

In some aspects, the subject can be at risk for developing heart failure or a type of heart failure. In some aspects, the type of heart failure can be preserved ejection fraction (HFpEF) or reduced ejection fraction (HFrEF).

The therapeutic agent, agent or treatment described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with a condition, disorder or disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of the cells described herein can be an amount that achieves a cure, but that outcome is only one among several that can be achieved. One or more of the symptoms can be less severe. Recovery can be accelerated in an individual who has been treated.

The therapeutically effective amount of the therapeutic agent, agent or treatment described herein and used in the methods as disclosed herein applied to mammals (e.g., humans) can be determined by one of ordinary skill in the art with consideration of individual differences in age, weight, and other general conditions.

The therapeutic agent, agent or treatment as described herein can be prepared for parenteral administration. The therapeutic agent, agent or treatment prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration.

### Examples

### Example 1: Genetic Architecture of Heart Failure with Preserved Versus Reduced Ejection Fraction

The two major clinical subtypes of heart failure (HF), heart failure with reduced ejection fraction (HFrEF) and heart failure with preserved ejection fraction (HFpEF), which are proposed to have different pathobiology based on epidemiological studies, clinical trials and pre-clinical investigations. Detailed genetic analyses was utilized to examine differences in pathobiology of HFrEF and HFpEF.

Utilizing clinical, laboratory and medication data combined with recorded left ventricular ejection fractions curated by natural language processing, cohorts of unclassified HF (n=43,344), HFrEF (n=19,495) and HFpEF (n=19,589) were created. Then a genome-wide association study (GWAS) for unclassified HF among non-Hispanic White participants in the Veterans Health Administration Million Veteran Program (n=258,943 controls without HF) was conducted and replicated and meta-analyzed discovery results in the United Kingdom Biobank. To further characterize the genetic determinants of each HF subtype, genetic correlation analyses between each clinical subtype of HF and HF risk factors, and Mendelian randomization association analyses of established HF risk factors with HFrEF and HFpEF was conducted.

Twenty genome-wide significant (GWS) loci was identified including ten novel loci for HF. Thirteen GWS loci associated with HFrEF, of which three novel loci (E2F6, PNMT and BPTF) were GWS in unclassified HF and HFrEF, while four novel loci (NFIA, MITF, PHACTR1 and METTL7A) were GWS in HFrEF was uncovered. One GWS locus was associated with HFpEF (FTO). Several loci were associated with known HF risk factors including type 2 diabetes, adiposity and systolic blood pressure; however, four HFrEF loci were not significantly associated with HF risk factors. Analyses of HF risk factors showed qualitative and quantitative differences in genetic correlation and Mendelian Randomization associations of HF risk factors with HFrEF and HFpEF.

The different genetic architecture of HFrEF and HFpEF indicates different pathophysiologic substrates underlying the two conditions. The finding of enhanced genetic discovery in HFrEF in spite of lower sample size as compared to modest genetic discovery in HFpEF in spite of similar sample size is likely due to the clinical heterogeneity of HFpEF, and indicates the need for improved phenotyping to facilitate mechanistic insights and efficacious interventions.

Methods. Datasets. Million Veteran Program: The design of MVP has been previously described (Gaziano J M, et al. J Clin Epidemiol. 2016; 70:214-23). Veterans were recruited from over 60 Veterans Health Administration (VA) medical centers nationwide since 2011. A feature of MVP is the linkage of a large biobank to an extensive, national, database from 2003 onward that integrates multiple elements such as diagnosis codes, procedure codes, laboratory values, and imaging reports, which permits detailed phenotyping of this large cohort. The MVP participants were genotyped as part of the study design.

UK Biobank: UK Biobank is a prospective study with over 500,000 participants aged 40-69 years recruited in 2006-2010 with extensive phenotypic and genotypic data (Bycroft C, Freeman C, et al. Nature. 2018; 562:203-209).

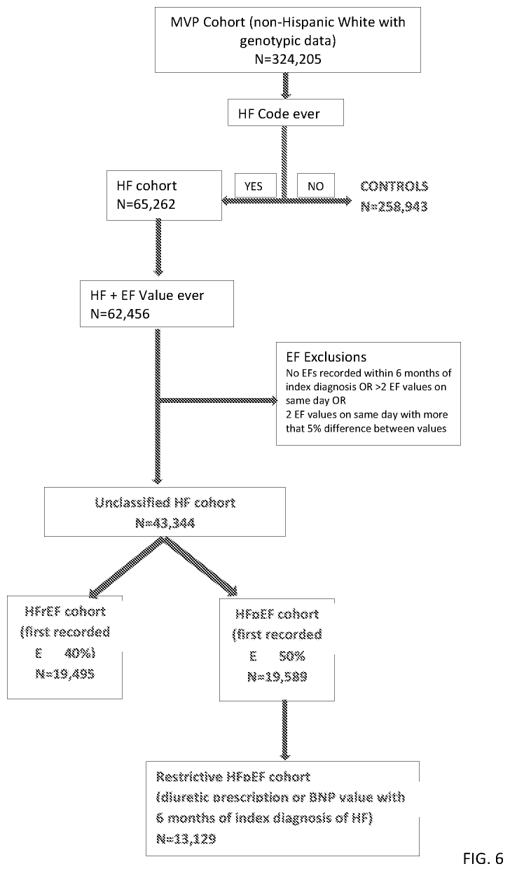

Phenotyping of Heart Failure, HFrEF, and HFpEF. HF patients from the MVP cohort were identified and classified into HFrEF and HFpEF (Patel Y R, et al. BMC Cardiovasc Disord. 2018; 18:128; Patel Y R, et al. J Am Heart Assoc. 2018; 7; and Kurgansky K E, et al. BMC Cardiovasc Disord. 2020; 20:92). As shown in FIG. 6, HF patients were identified as those with an International Classification of Diseases (ICD)-9 code of 428.x or ICD-10 code of 150.x and an echocardiogram performed within 6 months of diagnosis (median time period from diagnosis to echocardiography was 3 days, interquartile range 0-32 days). The requirement for echocardiogram improved the specificity of HF diagnosis. The index diagnosis of HF was documented during an outpatient encounter in the majority of cases. A natural language processing tool that was developed and validated in the national VA database to extract LVEF values from the VA Text Integration Utilities documents was utilized which included echocardiography reports, nuclear medicine reports, cardiac catheterization reports, history and physical examination notes, progress notes, discharge summary notes, and other cardiology notes, to ensure that we captured LVEF values measured outside the VA (Patel Y R, et al. BMC Cardiovasc Disord. 2018; 18:128; Patel Y R, et al. J Am Heart Assoc. 2018; 7; Patterson O V, et al. BMC Cardiovasc Disord. 2017; 17:151; and Freiberg M S, et al. JAMA Cardiol. 2017; 2:536-546). The accuracy of the LVEF value recordings has been validated (Patel Y R, et al. BMC Cardiovasc Disord. 2018; 18:128). A wider time frame between HF diagnosis and first recorded LVEF was utilized. HFpEF was classified as presence of HF diagnostic code and first recorded EF of ≥50% and HFrEF as HF diagnostic code with first recorded LVEF of ≤40%, to conform to current guidelines (Bozkurt B, et al. J Card Fail. 2021). In addition, a more restrictive definition of HFpEF was used with additional criteria of either prescription of diuretics or measurement of natriuretic peptides within 6 months of index diagnosis code for HF for confirming the presence of clinical HF which had a positive predictive value of 96%. Genetic associations between the HFpEF cohort was compared to the subset of HFpEF with more restrictive criteria to ensure that the genetic associations were similar between the two groups. Comorbid conditions were curated using ICD-10 or ICD-9 codes (Patel Y R, et al. J Am Heart Assoc. 2018; 7) In the UK Biobank, HF was defined as the presence of self-reported HF/pulmonary edema or cardiomyopathy at any visit; or an ICD-10 or ICD-9 billing code indicative of heart/ventricular failure or a cardiomyopathy of any cause, consistent with that used in a recent, international collaborative effort (Shah S, et al. Nat Commun. 2020; 11:163; and Aragam K G, et al. Circulation. 2018). Assessments of LVEF were not available in the majority of UK Biobank participants to permit classification into HFpEF and HFrEF.

Genetic Data Production, Quality Control and Imputation DNA extracted from participants' blood was genotyped using a customized Affymetrix Axiom® biobank array, the MVP 1.0 Genotyping Array. The array was enriched for both common and rare genetic variants of clinical significance in different ethnic backgrounds. Quality-control procedures were used to assign ancestry, remove low-quality samples and variants, and perform genotype imputation (Hunter-Zinck H, et al. Am J Hum Genet. 2020; 106:535-548). The following were excluded: duplicate samples, samples with more heterozygosity than expected, an excess (>2.5%) of missing genotype calls, or discordance between genetically inferred sex and phenotypic gender (Hunter-Zinck H, et al. Am J Hum Genet. 2020; 106:535-548). In addition, one individual from each pair of related individuals (more than second degree relatedness as measured by the KING software) (Manichaikul A, et al. Bioinformatics. 2010; 26:2867-73) were removed. Prior to imputation, variants that were poorly called (genotype missingness >5%) or that deviated from their expected allele frequency observed in the 1000 Genomes reference data were excluded. After pre-phasing using EAGLE v2.4, genotyped SNPs were imputed to the 1000 Genomes phase 3 version 5 reference panel using Minimac4. Imputed variants with poor imputation quality (r2<0.3) were excluded from further analyses.

Assignment of Racial Ethnic Groups in the MVP. The MVP participants were assigned to mutually exclusive racial/ethnic groups using HARE (Harmonized Ancestry and Race/Ethnicity), a machine learning algorithm that integrates genetically inferred ancestry (GIA) with self-identified race/ethnicity (SIRE) (Fang H, et al. Am J Hum Genet. 2019; 105:763-772). Briefly, HARE uses GIA to refine SIRE for genetic association studies in three ways: identify individuals whose SIRE are likely inaccurate, reconcile conflicts among multiple SIRE sources, and impute missing racial/ethnic information when the predictive confidence is high. GIA was inferred by computing top 30 PCs using flashPCA24 on an extended genotype dataset that included the MVP participants and an additional 2,504 individuals from the 1000 Genomes Phase 3 data. HARE assigned >98% of participants with genotype data to one of four non-overlapping groups: non-Hispanic European (EUR), non-Hispanic African (AFR), Hispanic (HIS), and non-Hispanic Asian Americans (ASN). The present GWAS of HF and subtypes focused on the MVP EUR group. The significant loci were examined in the AFR group.

Genome-wide Association Analysis. Imputed and directly measured genetic variants were tested for association assuming an additive genetic model using PLINK2 (Chang C C, et al. Gigascience. 2015; 4:7). The GWAS scan included variants with minor allele frequency higher than 1%. Logistic regression of HF, HFrEF and HFpEF was adjusted for age, sex, and the top ten genotype-derived principal components. In UK Biobank analyses, an additional covariate was included for genotyping array. The GWAS results of HF from MVP and UK Biobank were meta analyzed using inverse-variance weighted fixed-effects model implemented in METAL (Willer C J, Li Y and Abecasis G R. Bioinformatics. 2010; 26:2190-1). Joint meta-analysis results were reported for unclassified HF to improve the power for GWAS discovery (Skol A D, et al. Nat Genet. 2006; 38:209-13). GWAS results were summarized using FUMA, a platform that annotates, prioritizes, visualizes and interprets GWAS results (Watanabe K, et al. Nat Commun. 2017; 8:1826). Genome-wide significant SNPs (P<5×10−8) were grouped into a genomic locus based on either r2>0.1 or distance between loci of <500 kb using the 1000 Genomes European reference panel. Lead SNPs were defined within each locus if they were independent (r2<0.1). Loci were considered novel if the sentinel SNP was of genome-wide significance (P<5×10−8) and located >1 Mb from previously reported GWS SNPs associated with HF (Shah S, et al. Nat Commun. 2020; 11:163; and Aragam K G, et al. Circulation. 2018). For novel loci, the genomic base-pair position of each sentinel SNP was used to map to the closest gene within a 500 kb region as the candidate gene. The physical base-pair location (GRCh37/hg19) and alleles were used to uniquely identify a genetic variant to replicate previous reported genetic associations with HF, and with HF risk factors.

For replication, genome-wide association testing was conducted among UK Biobank participants passing sample quality control, comparing unclassified HF cases with non-HF controls. Procedures for genotyping and genotype imputation in the UK Biobank have been described previously (Bycroft C, et al. Nature. 2018; 562:203-209). For genetic association testing, SNPs with minor allele frequency (MAF) >1% available in the Haplotype Reference Consortium (HRC), and imputation quality (INFO) >0.3 were included. The analyses was restricted to samples of European genetic ancestry, defined by a combination of self-reported race and genetic principal components of ancestry. Specifically, samples with genetic data who self-reported as white (British, Irish, or Other) and applied an outlier detection protocol (R package aberrant) to three pairs of principal components (PC1/PC2, PC3/PC4, and PC5/PC6) were selected, as generated centrally by the UK Biobank. Outliers in any of the three pairs of PCs were excluded from analysis to ensure that the study population was relatively homogenous in terms of genetic ancestry. Additional sample exclusions were implemented for 2nd-degree or closer relatedness (Kinship coefficient >0.0884), sex chromosome aneuploidy, and excess missingness or heterozygosity, as defined by the UK Biobank. Association analyses were performed using PLINK2 (cog-genomics.org/plink/2.0/) (Chang C C, et al. Gigascience. 2015; 4:7) on imputed genotype dosages, and a logistic regression model was used adjusting for age at enrollment, sex, genotyping array, and the first 10 principal components of ancestry. After merging with the phenotypic data, a total of 8,227 unclassified HF cases were comparted to 379,788 non-HF controls. Test statistic inflation was investigated by genomic control and inspection of quantile-quantile plots.

Genetic Correlation. Genetic correlation is an important population parameter that describes the shared genetic architecture of complex traits. Using the SNP-heritabilities, cross-trait LD Score Regression (LDSC) (Bulik-Sullivan B K, et al., Nat Genet. 2015; 47:291-5; and Ni G, et al. Am J Hum Genet. 2018; 102:1185-1194) estimates genetic correlation that requires GWAS summary statistics and is not biased by sample overlap. Genetic correlation between HF, HFrEF and HFpEF, and risk factors were estimated using LDSC and European ancestry-based GWAS results of these traits. A reference panel consisting of 1.2 million HapMap3 variants was used to merge with GWAS summary statistics filtered to variants with MAF >0.01, Hardy-Weinberg equilibrium (HWE) P>10-20 and imputation R2>0.5. Using LDSC and GWAS summary statistics, the inflation factor of composite HF, HFpEF and HFrEF was also estimated.

Mendelian Randomization Analysis of HF Risk Factors. Two-sample Mendelian Randomization (MR) was conducted to examine for possible causal associations using multiple genetic instrumental variables from previous GWAS of HF risk factors including coronary artery disease (CAD) (Nikpay M, et al. Nat Genet. 2015; 47:1121-1130), atrial fibrillation (AFib) (Roselli C, et al. Nat Genet. 2018; 50:1225-1233), type 2 diabetes (T2D) (Scott R A, et al. Diabetes. 2017; 66:2888-2902), body mass index (BMI) (Locke A E, et al. Nature. 2015; 518:197-206.), lipids (Willer C J, et al. Nat Genet. 2013; 45:1274-1283), blood pressure (Warren H R, et al. Nat Genet. 2017; 49:403-415) and estimated glomerular filtration rate (eGFR) (Pattaro C, et al. Nat Commun. 2016; 7:10023). The GWS sentinel SNPs from each GWAS were selected as the genetic instrumental variables (GIVs) for each HF risk factor. The MR association of each risk factor was estimated using three complementary methods: inverse-variance-weighted (IVW), median weighted, and MR-Egger regression, as implemented in the R package TwoSampleMR (Hemani G, et al. Elife. 2018; 7). MR-Egger regression was used to identify the horizontal pleiotropy measured by the intercept of the regression. Random-effects model was used to estimate the MR association between HF risk factors and HF outcomes for IVW and MR-Egger regression. To avoid sample overlap in the two-sample MR design, summary statistics of Composite HF, HFrEF and HFpEF from the MVP study was used, and summary statistics of risk factors in previous GWAS without the MVP was also used, all from studies of European ancestry. Nominal p-value of 0.05 was considered as suggestive evidence for MR association for each HF risk factor. A stringent Bonferroni correction was applied for 12 tested factors (p-value<0.05/12=0.0042) acknowledging that some factors are not independent.

Comorbidities were curated from the national databases using the International Classification of Diseases (ICD)-10 and ICD-9 codes. The codes used to extract each comorbidity are listed below (% sign indicates that any code with the preceding number was utilized).

### Atrial Fibrillation:

ICD-9: 427.310%
ICD-10: 148.2%, 148.0%, 148.91%, 148.1%

### Coronary Artery Disease:

ICD-9: 410%, 411%, 412%, 413%, 414%
ICD-10: 120%, I21%, I22%, I23%, I24%, I25%

### Chronic Kidney Disease:

ICD-9: 403.01%, 404.02%, 404.10%, 585.00%, 403.11%, 404.03%, 404.90%, 586.00%, 403.91%, 404.10%, 584.00%, 792.50%
ICD-10: V42.0%, V56.0%, V56.3%, N18%, V45.1%, V56.1%, V56.8%, V56.2%

### Diabetes Mellitus:

ICD-9: 250%
ICD-10: E08%, E09% y, E10%, E11%, E12%, E13%

### Hyperlipidemia:

### ICD-9: 272%

ICD-10: E78.0, E75.249, E78.01, E77.0, E78.1, E77.1, E78.2, E78.81, E78.3, E78.89, E78.4, E78.9, E78.5, E88.1, E78.6, E88.89, E75.21, E75.22

### Hypertension:

ICD-9: 401%, 402%, 403%, 404%, 405%
ICD-10: 110%, 111%, 112%, 113%, 115%, 116%, 167.4%

### Peripheral Vascular Disease:

ICD-9: 440%, 441%, 442%, 443%, 444%, 447%, 451%, 452%, 453%, 557%
ICD-10: E08.51, E08.52, E09.51, E09.52, E10.51, E10.52, E11.51, E11.52, E13.51, E13.52, I67.0, I70.0, I70%, 171%, 172%, 173.01, I73.1, I73.81, I73.89, I73.9, I74.01, I74.09, I74.10, I74.11, I74.19, I74.2, I74.3, I74.4, I74.5, I74.8, I74.9, I77%, I79.0, I79.1, I79.8, I80%, I81, I82%, K55%, K76.5, M31.8, M31.9

### Stroke or Transient Ischemic Attack:

ICD-9: 430%, 431%, 433%, 434%, 436%, 437.0%, 437.6%
ICD-10: I63%, I65%, I66%, I67.2%, I67.8%, I67.6%, I60%, I61%, G45%

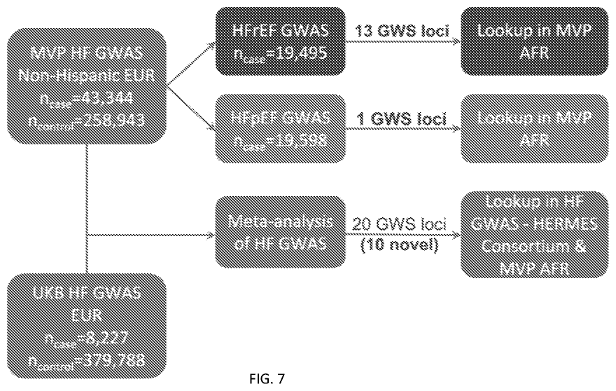

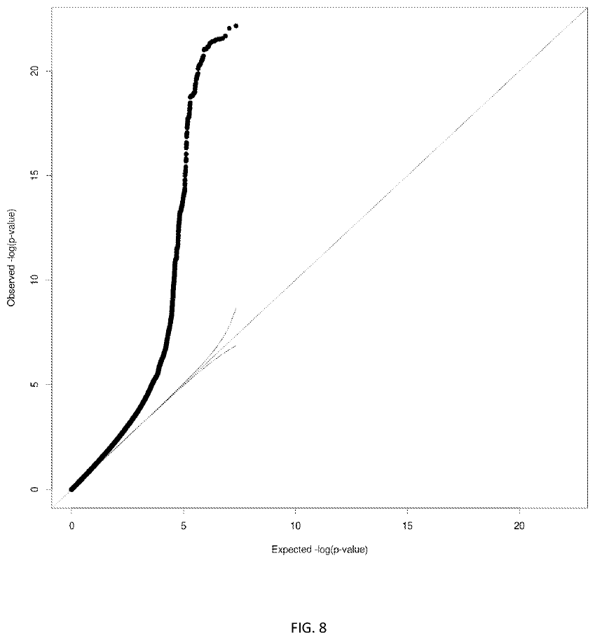

Results. The primary study population for the GWAS consisted of 258,943 controls, and cases of HF (n=43,344), HFpEF (n=19,589), and HFrEF (n=19,495) from the MVP cohort, and 8,227 HF cases and 379,788 controls from the UK Biobank cohort, each with European ancestry. The GWS associations of unclassified HF, HFrEF and HFpEF were then examined in the MVP non-Hispanic African Americans (AFR) and a recent HF GWAS from the HERMES consortium (FIG. 7). The MVP control and HF cohorts were predominantly male. In both MVP and UK Biobank, the HF cohorts tended to be older with a higher prevalence of cardiometabolic risk factors and comorbidities than the control populations (Tables 1, 2, and 3).

TABLE 1
Characteristics of HF Patients and non-HF Controls
in the MVP Participants of European Ancestry.

| | | | | Unclassified |
|---|---|---|---|---|
| | Control | HFpEF | HFrEF | HF |
| Group | (N = 258,943) | (N = 19,589) | (N = 19,495) | (N = 43,344) |
| Age (years), mean ± SD | 62.74 ± 13.76 | 69.88 ± 9.77 | 69.29 ± 9.74 | 69.61 ± 9.74 |
| Male (%) | 92.14 | 95.74 | 97.85 | 96.92 |
| Body mass index (kg/m²), | 29.20 ± 5.53 | 31.95 ± 6.98 | 30.20 ± 6.38 | 31.08 ± 6.73 |
| mean ± SD | | | | |
| Underweight (<18.5) % | 0.56 | 0.47 | 0.59 | 0.52 |
| Normal (18.5-24.9) % | 20.25 | 13.43 | 18.79 | 16.05 |
| Overweight (25.0-29.9) % | 40.66 | 29.71 | 35.09 | 32.44 |
| Obese (30.0-34.9) % | 24.70 | 27.08 | 25.62 | 26.37 |
| Morbidly obese (≥35.0) % | 13.84 | 29.31 | 19.91 | 24.62 |
| Mean LVEF, mean ± SD | NA | 56.97 ± 5.65 | 29.33 ± 9.36 | 43.36 ± 15.05 |
| Atrial fibrillation (%) | 6.33 | 30.80 | 37.83 | 34.44 |
| Coronary artery disease (%) | 22.47 | 63.87 | 74.63 | 69.72 |
| Chronic kidney disease (%) | 9.54 | 37.21 | 35.75 | 36.43 |
| Diabetes (%) | 20.61 | 48.54 | 45.06 | 46.76 |
| Hyperlipidemia (%) | 66.9 | 87.75 | 88.20 | 88.04 |
| Hypertension (%) | 62.97 | 93.22 | 91.69 | 92.51 |
| Peripheral vascular disease (%) | 15.18 | 42.47 | 42.27 | 42.47 |
| Stroke/TIA (%) | 8.26 | 25.29 | 24.33 | 24.93 |

HFpEF: heart failure with preserved ejection fraction; HFrEF: heart failure with reduced ejection fraction; HF: heart failure; SD: standard deviation; LVEF: left ventricular ejection fraction; TIA: transient ischemic attack.

TABLE 2
Characteristics of non-HF controls and HF cases including clinical subtypes in the
MVP cohort of non-Hispanic European (EUR) and non-Hispanic African Americans (AFR).

| | MVP EUR | | | | |
|---|---|---|---|---|---|
| | | | | HF with | |
| | | | | mid-range | Unclassified |
| | Control | HFpEF | HFrEF | EF | HF |
| | (N = 258,943) | (N = 19,589) | (N = 19,495) | (N = 4,260) | (N = 43,344) |
| Age (years), mean ± SD | 62.74 ± 13.76 | 69.88 ± 9.77 | 69.29 ± 9.74 | 69.84 ± 9.59 | 69.61 ± 9.74 |
| Male (%) | 92.14 | 95.74 | 97.85 | 98.12 | 96.92 |
| Body mass index (kg/m2), | 29.20 ± 5.53 | 31.95 ± 6.98 | 30.20 ± 6.38 | 31.04 ± 6.55 | 31.08 ± 6.73 |
| mean ± SD | | | | | |
| Underweight (<18.5) % | 0.56 | 0.47 | 0.59 | 0.42 | 0.52 |
| Normal (18.5-24.9) % | 20.25 | 13.43 | 18.79 | 15.56 | 16.05 |
| Overweight (25.0-29.9) % | 40.66 | 29.71 | 35.09 | 32.89 | 32.44 |
| Obese (30.0-34.9) % | 24.7 | 27.08 | 25.62 | 26.57 | 26.37 |
| Morbidly obese (≥35.0) % | 13.84 | 29.31 | 19.91 | 24.56 | 24.62 |
| Mean LVEF, mean ± SD | NA | 56.97 ± 5.65 | 29.33 ± 9.36 | 45.01 ± 1.82 | 43.36 ± 15.05 |
| Atrial fibrillation %) | 6.33 | 30.8 | 37.83 | 35.7 | 34.44 |
| Coronary artery disease (%) | 22.47 | 63.87 | 74.63 | 74.19 | 69.72 |
| Chronic kidney disease (%) | 9.54 | 37.21 | 35.75 | 35.93 | 36.43 |
| Diabetes (%) | 20.61 | 48.54 | 45.06 | 46.34 | 46.76 |
| Hyperlipidemia (%) | 66.9 | 87.75 | 88.2 | 88.61 | 88.04 |
| Hypertension (%) | 62.97 | 93.22 | 91.69 | 92.96 | 92.51 |
| Peripheral vascular disease | 15.18 | 42.47 | 42.27 | 43.38 | 42.47 |
| (%) | | | | | |
| Stroke/TIA (%) | 8.26 | 25.29 | 24.33 | 26.06 | 24.93 |

HFpEF:heart failure with preserved ejection fraction; HFrEF:heart failure with reduced ejection fraction; HF:heart failure; SD:standard deviation; LVEF:left ventricular ejection fraction; TIA:transient ischemic attack.

TABLE 3
Characteristics of non-HF controls and HF cases
in the UK Biobank cohort of European ancestry.

| | | | Heart Failure | | | |
|---|---|---|---|---|---|---|
| | | | Cases | | Controls | |
| | All | | (N = 8,227) | | (N = 379,788) | |
| Age (SD) | 56.9 | (8.0) | 62.0 | (6.2) | 56.8 | (8.0) |
| Men, N (%) | 179,804 | (46.33) | 5,585 | (67.9) | 174,152 | (45.9) |
| Body mass index (SD) | 27.4 | (4.8) | 29.8 | (5.8) | 27.4 | (4.7) |
| Hypertension, N (%) | 126,813 | (32.7) | 5,980 | (72.7) | 120,779 | (31.8) |
| Coronary Artery Disease, N (%) | 27,928 | (7.2) | 4,540 | (55.2) | 2,330 | (6.2) |
| Chronic Kidney Disease, N (%) | 5,130 | (1.3) | 1,090 | (13.3) | 4,032 | (1.1) |
| Atrial Fibrillation, N (%) | 15,024 | (3.9) | 3,200 | (38.9) | 11,775 | (3.1) |
| Type 2 Diabetes, N (%) | 18,258 | (4.7) | 1,886 | (22.9) | 16,364 | (4.3) |
| Peripheral Vascular Disease, N | 4,726 | (1.2) | 781 | (9.5) | 3,940 | (1.0) |
| (%) | | | | | | |
| Hypercholesterolemia, N (%) | 68,301 | (17.6) | 4,227 | (51.4) | 64,035 | (16.9) |

SD:standard deviation

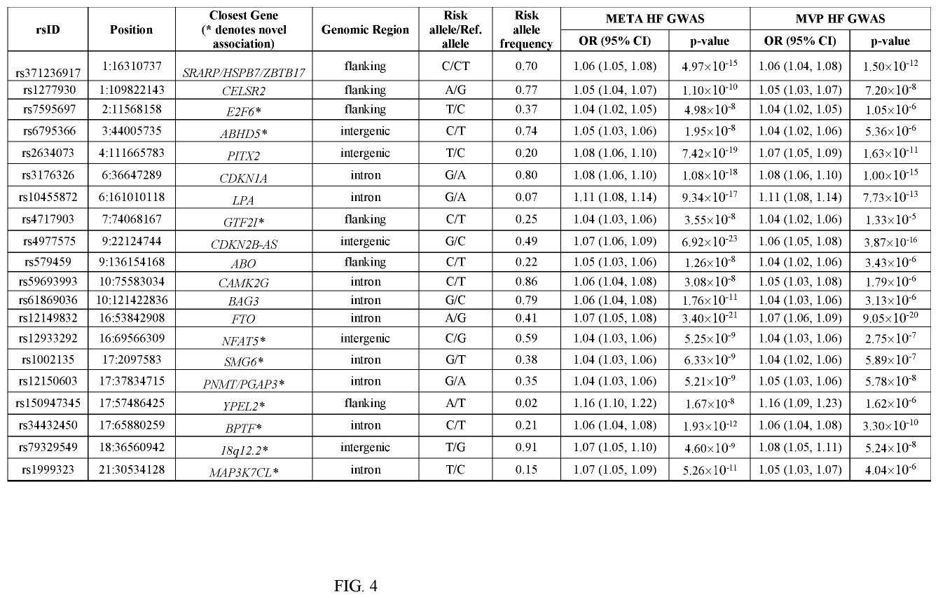

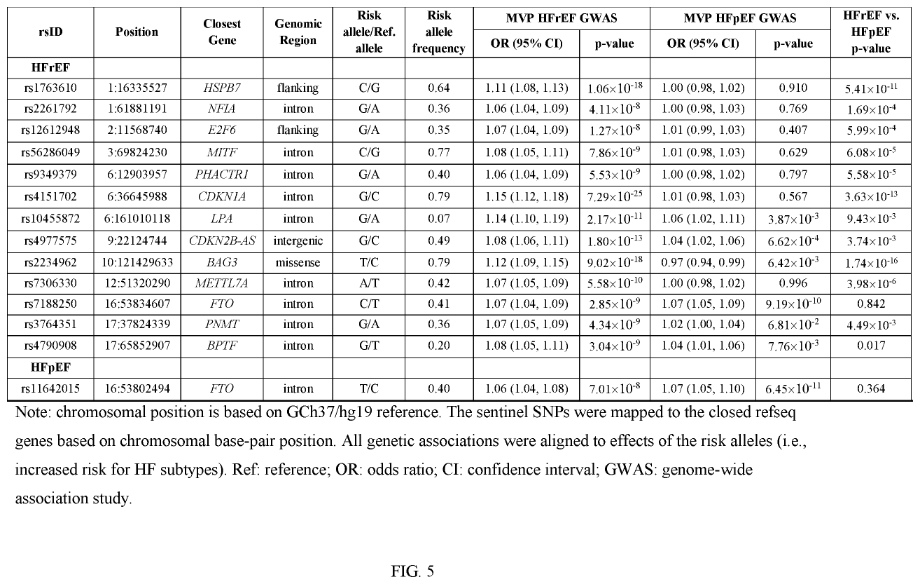

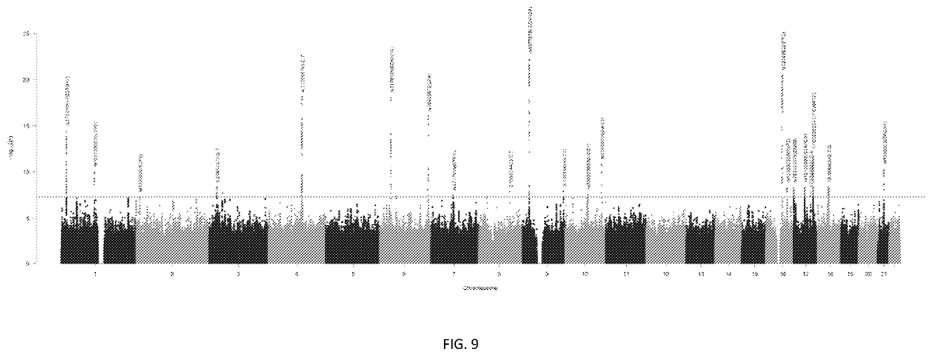

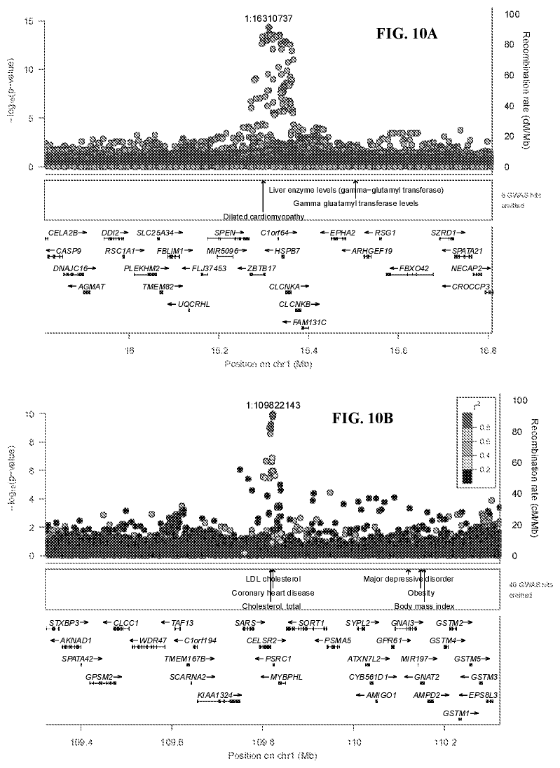

GWAS of Unclassified HF. In unclassified HF, the meta-analysis of MVP and UKB GWAS results (FIGS. 8 and 9) identified 20 genome-wide significant (GWS) loci including 10 novel loci (FIG. 4). The regional association plots of each GWS locus are shown in FIGS. 10A-5T. Twelve GWS independent SNPs associated with HF from a recent HF GWAS publication (Shah S, et al. Nat Commun. 2020; 11:163), and three out of four previously reported associations for dilated cardiomyopathy (DCM), an established cause of HFrEF (Stark K, et al. PLoS Genet. 2010; 6:e1001167; Villard E, et al. Eur Heart J. 2011; 32:1065-76; and Meder B, et al. Eur Heart J. 2014; 35:1069-77) (Bonferroni-corrected p-value <0.05 Table 4) was replicated.

TABLE 4
Replication of previous reported variants associated with
composite heart failure or dilated cardiomyopathy.

| | | | Risk/ref | |
|---|---|---|---|---|
| rsID | MarkerName | Phenotype | allele | Nearest Genes |
| rs10927875 | 1:16299312:C:T | Dilated | C/T | ZBTB17 |
| | | cardiomyopathy | | |
| rs1739843 | 1:16343254:C:T | Dilated | C/T | HSPB7 |
| | | cardiomyopathy | | |
| rs1739843 | 1:16343254:C:T | Heart Failure | C/T | HSPB7 |
| rs660240 | 1:109817838:C:T | Heart Failure | C/T | CELSR2 |
| rs6787362 | 3:69227379:A:G | Heart Failure | G/A | FRMD4B |
| rs1906609 | 4:111666451:G:T | Heart Failure | T/G | PITX2 |
| rs17042102 | 4:111668626:G:A | Heart Failure | A/G | PITX2, |
| | | | | FAM241A |
| rs11745324 | 5:137012171:G:A | Heart Failure | G/A | KLHL3 |
| rs9262636 | 6:31025848:A:G | Dilated | G/A | HCG22 |
| | | cardiomyopathy | | |
| rs4135240 | 6:36647680:T:C | Heart Failure | T/C | CDKNIA |
| rs55730499 | 6:161005610:C:T | Heart Failure | T/C | LPA |
| rs140570886 | 6:161013013:T:C | Heart Failure | C/T | LPA |
| rs2291569 | 7:128488734:G:A | Dilated | G/A | FLNC |
| | | cardiomyopathy | | |
| rs1556516 | 9:22100176:C:G | Heart Failure | C/G | 9p21/CDKN2B- |
| | | | | AS1 |
| rs600038 | 9:136151806:T:C | Heart Failure | C/T | ABO, SURF1 |
| rs4746140 | 10:75417249:G:C | Heart Failure | G/C | SYNPO2L, |
| | | | | AGAP5 |
| rs2234962 | 10:121429633:T:C | Dilated | T/C | BAG3 |
| | | cardiomyopathy | | |
| rs4766578 | 12:111904371:A:T | Heart Failure | T/A | ATXN2 |
| rs10519210 | 15:63737925:T:G | Heart Failure | G/T | USP3 |
| rs56094641 | 16:53806453:A:G | Heart Failure | G/A | FTO |

The genetic associations were aligned to effects of the risk alleles (i.e., increased risk for unclassified HF).

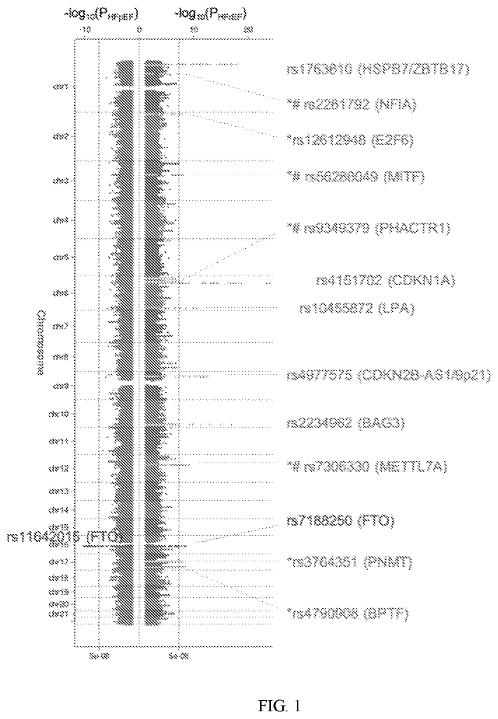

GWAS of HFrEF and HFpEF. In the GWAS among the MVP participants of European ancestry, 13 GWS loci associated with HFrEF and one GWS locus (FTO) associated with HFpEF (FIG. 1; FIG. 5; and FIGS. 11A and 111B) was identified. The regional association plots of each GWS locus are shown in FIGS. 12A-12N. Two lead SNPs in the FTO locus for HFrEF (rs7188250) and HFpEF (rs11642015) were in linkage disequilibrium (r2=0.873). Among these thirteen loci associated with HF subtypes, seven loci (NFIA, E2F6, MITF, PHACTR1, METTL7A, PNMT and BPTF) have not been reported in previous HF-related GWAS, of which four loci (NFIA, MITF, PHACTR1 and METTL7A) were GWS only in GWAS of HFrEF cases. Using LDSC, we identified a modest positive genetic correlation between HFrEF and HFpEF (0.57±0.07) comparing to higher genetic correlation between HFrEF and unclassified HF (0.92±0.02), and between HFpEF and unclassified HF (0.84±0.03).

It was observed that moderate genomic inflation (k for unclassified HF (GC k=1.263), HFrEF (GC k=1.152) and HFpEF (GC k=1.118), which were typical in GWAS with large sample sizes. The LDSC intercepts were 1.044 (SE 0.010), 1.013 (SE 0.008) and 1.028 (SE 0.008) for unclassified HF, HFrEF and HFpEF, respectively, indicating the majority of the inflation was due to polygenicity of HF and subtypes.

Replication in MVP African Americans and other HF GWAS. Among 20 GWS loci identified in the GWAS of unclassified HF in the European ancestry, the associations of the sentinel SNPs in the MVP African Americans and a recently reported HF GWAS was checked. Among MVP African Americans, all but two SNPs had genetic associations with composite HF in the same direction, and two were significant after Bonferroni correction of 20 SNP tests (p-value<2.5×10−3), including rs3176326 (CDKN1A, OR 1.07, 95% CI 1.03-1.12), p-value 7.65×10−4) and rs12150603 (PNMT, OR 1.09, 95% CI 1.05-1.13, p-value 5.28×10−6). The GWS sentinel SNPs had the same direction of effects in the HF GWAS conducted by the HERMES consortium. Among ten novel loci of unclassified heart failure, four were replicated after Bonferroni correction of 20 SNP tests (p-value<2.5×10−3), including rs4717903 (GTF2I, OR 1.03, 95% CI 1.02-1.05, p-value 2.24×10−4), rs12933292 (NFAT5, OR 1.03, 95% CI 1.01-1.04, p-value 4.32×10−4), rs1002135 (SMG6, OR 1.03, 95% CI 1.01-1.04, p-value 6.67×10−4) and rs1999323 (MAP3K7CL, OR 1.04, 95% CI 1.02-1.07, p-value 5.39×10−5).

Among 13 GWS loci associated with HFrEF, 11 had genetic effects in the same direction in the MVP African American cohort (Table 5), including three which were test-wise significant after Bonferroni correction (p-value<3.8×10−3). Interestingly, the sentinel SNP of the FTO locus was significantly associated with HFpEF (rs11642015, OR 1.10, 95% CI 1.03-1.17, p-value 6.30×10−3), but not associated with HFrEF (rs7188250, OR 1.06, 95% CI 0.99-1.12, p-value 0.11) in the MVP African Americans.

TABLE 5
Summary of genome-wide significant loci and sentinel
variants associated with HFrEF or HFpEF.

| | | | | Risk |
|---|---|---|---|---|
| | | | Genomic | allele/Ref. |
| rsID | Position | Closest Gene | Region | allele |
| HFrEF | | | | |
| rs1763610 | 1:16335527 | HSPB7 | flanking | C/G |
| rs2261792 | 1:61881191 | NFIA | intron | G/A |
| rs12612948 | 2:11568740 | E2F6 | flanking | G/A |
| rs56286049 | 3:69824230 | MITF | intron | C/G |
| rs9349379 | 6:12903957 | PHACTRI | intron | G/A |
| rs4151702 | 6:36645988 | CDKN1A | intron | G/C |
| rs10455872 | 6:161010118 | LPA | intron | G/A |
| rs4977575 | 9:22124744 | CDKN2B-AS | intergenic | G/C |
| rs2234962 | 10:121429633 | BAG3 | missense | T/C |
| rs7306330 | 12:51320290 | METTL7A | intron | A/T |
| rs7188250 | 16:53834607 | FTO | intron | C/T |
| rs3764351 | 17:37824339 | PNMT | intron | G/A |
| rs4790908 | 17:65852907 | BPTF | intron | G/T |
| HFpEF | | | | |
| rs11642015 | 16:53802494 | FTO | intron | T/C |

Bold font indicates significant associations after Bonferroni correction for multiple testing (p<0.0038). Chromosomal position is based on GCh37/hg19 reference. The sentinel SNPs were mapped to the closed refseq genes based on chromosomal base-pair position. The genetic associations were aligned to effects of the risk alleles (i.e., increased risk for HF subtypes).

Genetic Associations with HFrEF and HFpEF in Candidate Genes and Loci. The associations of all 12 GWS loci reported in the recent HERMES study with HFrEF were replicated, but four variants were significantly associated with HFpEF including the FTO locus (Table 4). Other loci replicated in HFrEF were ZBTB171HSPB7 locus (closest gene of SRARP discovered in this study) and HCG22 locus 41 (OR 1.05, CI 1.03-1.08, P=7.83×10−5). The genetic effect sizes were also larger for HFrEF than that for HF (e.g., rs1739843 for HFrEF: OR 1.08, CI 1.06-1.10, P=3.47×10−12; for HF OR 1.04, CI 1.03-1.05, P=2.27×10−8). Reported associations of FRMD4B or USP3 region with HF could not be replicated (Smith N L, et al. Circ Cardiovasc Genet. 2010; 3:256-66; and Cappola T P, et al. Circ Cardiovasc Genet. 2010; 3:147-54). Regardless of the significance thresholds, the previous reported HF-associated variants had the same direction of effects across HF, HFrEF and HFpEF (Table 4).

Among 15 autosomal genes related to cardiomyopathy (Kalia S S, et al. Genet Med. 2017; 19:249-255), one gene, TMEM43, was found to harbor significant genetic associations of common variants with HFrEF (lowest P=5.06×10−8, MAF=0.32; FIG. 13).

Among 13 HFrEF-associated loci, nine loci were differentially associated between HFrEF and HFpEF (p-value<0.0038, corrected for 13 tests). For example, the risk allele of the BAG3 missense variant (rs2234962) was associated with higher risk for HFrEF (OR 1.12, 95% CI 1.09-1.15, p-value 9.02×10−18), but was associated with lower risk for HFpEF (OR 0.97, 95% CI 0.94-0.99, p-value 6.42×10−3). Four loci, including LPA, FTO, PNMT and BPTF, were not differentially associated with HF subtypes. The FTO locus was consistently associated with both HFrEF and HFpEF.

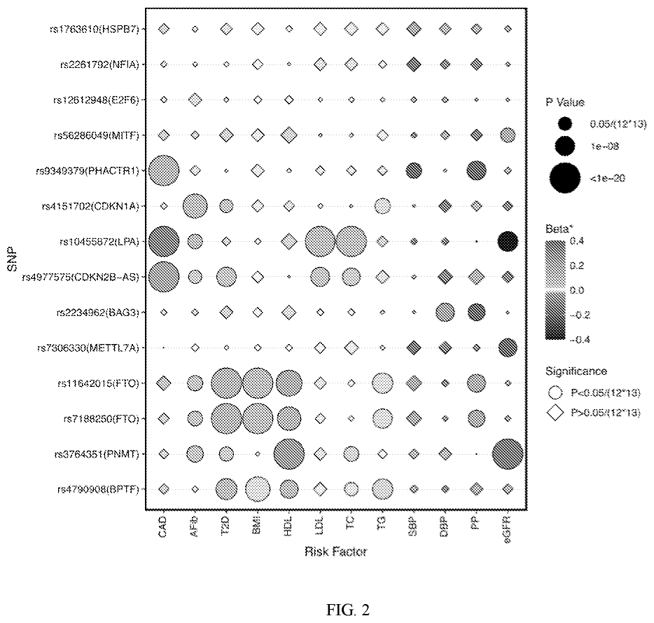

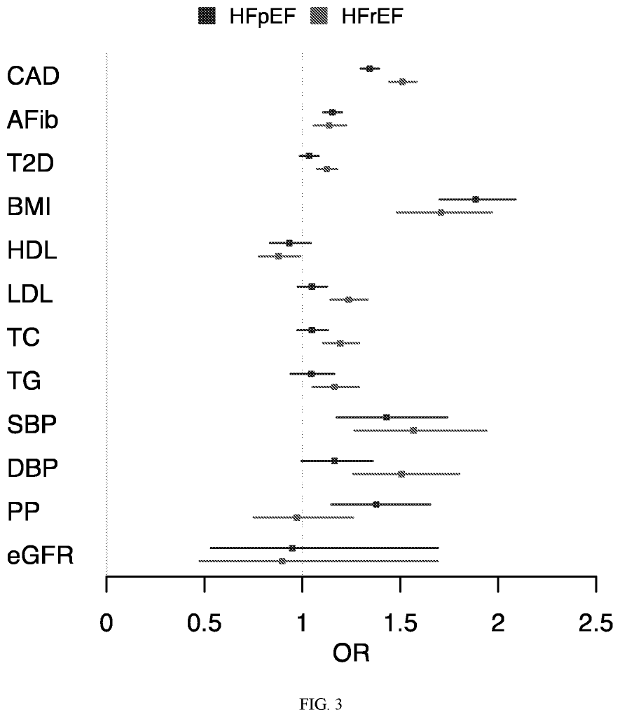

Associations of HFrEF- and HFpEF Loci with Cardiovascular Risk Factors. As shown in FIG. 2 and Table 6, several of the 13 loci associated with HFrEF and HFpEF also demonstrated genetic associations with risk factors as previously reported (PHACTR1, LPA, and CDKN2B-AS with CAD; CDKN1A with AF); and FTO with BMI, T2D, and HDL cholesterol). Although most loci were associated with multiple risk factors, the BAG3 locus was associated with blood pressure traits, and the MITF and METTL7A loci are associated with eGFR. Three novel loci, SRARP, NFIA and E2F6, are not significantly associated with any tested HF risk factors.

TABLE 6
Genetic associations between HFrEF- and HFpEF-associated
sentinel variants and heart failure risk factors.

| | | | | | ALT |
|---|---|---|---|---|---|
| | | | | Effect | FREQ |
| ID | rsID | CHROM | Genes | Allele | MVP |
| 1:16335527:G:C | rs1763610 | 1 | SPEN/ZBTB17/ | C | 0.6448 |
| | | | Clorf64/HSPB7/ | | |
| | | | CLCNKA/CLCNKB | | |
| 1:61881191:G:A | rs2261792 | 1 | NFIA | G | 0.364538 |
| 2:11568740:A:G | rs12612948 | 2 | E2F6/AC099344.1 | G | 0.351196 |
| 3:69824230:C:G | rs56286049 | 3 | MITF | C | 0.772007 |
| 6:12903957:A:G | rs9349379 | 6 | PHACTRI | G | 0.40194 |
| 6:161010118:A:G | rs10455872 | 6 | SLC22A2/SLC22A3/ | G | 0.0688417 |
| | | | LPA/PLG | | |
| 6:36645988:G:C | rs4151702 | 6 | CDKN1A | G | 0.79303 |
| 9:22124744:G:C | rs4977575 | 9 | RP11-145E5.5/ | G | 0.492917 |
| | | | CDKN2A/CDKN2B | | |
| 10:121429633:T:C | rs2234962 | 10 | BAG3/INPP5F/ | T | 0.789251 |
| | | | MCMBP/SEC23IP | | |
| 12:51320290:T:A | rs7306330 | 12 | LIMA1/LARP4/ | A | 0.42207 |
| | | | DIP2B/ATF1/ | | |
| | | | TMPRSS12/ | | |
| | | | METTL7A | | |
| 16:53802494:C:T | rs11642015 | 16 | FTO | T | 0.403425 |
| 16:53834607:T:C | rs7188250 | 16 | FTO | C | 0.411651 |
| 17:37824339:G:A | rs3764351 | 17 | FBXL20/CDK12/ | G | 0.35852 |
| | | | NEUROD2/ | | |
| | | | PPPIRIB/STARD3/ | | |
| | | | TCAP/ | | |
| | | | PNMT/PGAP3/ | | |
| | | | ERBB2/MIEN1/ | | |
| | | | GRB7 | | |
| 17:65852907:T:G | rs4790908 | 17 | BPTF/C17orf58/ | G | 0.203441 |
| | | | KPNA2 | | |

A sensitivity analyses of HF, HFrEF and HFpEF was conducted by additionally adjusting for BMI or diabetes in the genetic association models. For composite HF, including BMI or diabetes as an additional covariate did not change the significant genetic associations except for the BMI adjustment in the FTO locus (7). The association of rs12149832 (ETO) was reduced from OR 1.07 (95% CI 1.06-1.09, p-value 9.05×10−20) to OR 1.04 (95% CI 1.02-1.05, p-value 2.23×10−6). Similar results were observed in the genetic association analyses of HFrEF and HFpEF (Table 8). Adjustment for diabetes did not affect any of the significant genetic associations with HFrEF and HFpEF. Adjustment for BMI reduced the genetic associations of sentinel SNPs in the ETO locus with HFrEF (rs7188250: from OR 1.07, 95% CI 1.04-1.09, p-value 2.85×10−9 to OR 1.04, 95% CI 1.02-1.07, p-value 5.72×10⁻⁵) and with HFpEF (rs 11642015: OR 1.07, 95% CI 1.05-1.1, p-value 6.45×10−11 to OR 1.02, 95% CI 1-1.04, p-value 0.045).

Tables 7A-C. Sensitivity analyses of genome-wide significant sentinel variants associated with unclassified HF additionally adjusted for BMI or diabetic status.

TABLE 7A
HF in MVP EUR.

| | | | | Risk | | HF in MVP EUR | | | |
|---|---|---|---|---|---|---|---|---|---|
| | | | | allele | Risk | | Lower | Upper | |
| | | Closest | Genomic | Ref | allele | | 95% | 95% | p- |
| Position | rsID. | Gene | Region | allele | freq. | OR | CI | CI | value |
| 1:16310737 | rs371236917 | SRARP/ | flanking | C/CT | 0.7 | 1.06 | 1.04 | 1.08 | 1.50E−12 |
| | | HSPB7/ | | | | | | | |
| | | ZBTB17 | | | | | | | |
| 1:109822143 | †rs1277930 | CELSR2 | flanking | A/G | 0.77 | 1.05 | 1.03 | 1.07 | 7.20E−08 |
| 2:11568158 | †rs7595697 | E2F6* | flanking | T/C | 0.37 | 1.04 | 1.02 | 1.05 | 1.05E−06 |
| 3:44005735 | †rs6795366 | ABHD5* | intergenic | C/T | 0.74 | 1.04 | 1.02 | 1.06 | 5.36E−06 |
| 4:111665783 | rs2634073 | PITX2 | intergenic | T/C | 0.2 | 1.07 | 1.05 | 1.09 | 1.63E−11 |
| 6:36647289 | rs3176326 | CDKN1A | intron | G/A | 0.8 | 1.08 | 1.06 | 1.1 | 1.00E−15 |
| 6:161010118 | rs10455872 | LPA | intron | G/A | 0.07 | 1.11 | 1.08 | 1.14 | 7.73E−13 |
| 7:74068167 | rs4717903 | GTF2I* | flanking | C/T | 0.25 | 1.04 | 1.02 | 1.06 | 1.33E−05 |
| 9:22124744 | †rs4977575 | CDKN2B-AS | intergenic | G/C | 0.49 | 1.06 | 1.05 | 1.08 | 3.87E−16 |
| 9:136154168 | rs579459 | ABO | flanking | C/T | 0.22 | 1.04 | 1.02 | 1.06 | 3.43E−06 |
| 10:75583034 | rs59693993 | CAMK2G | intron | C/T | 0.86 | 1.05 | 1.03 | 1.08 | 1.79E−06 |
| 10:121422836 | rs61869036 | BAG3 | intron | G/C | 0.79 | 1.04 | 1.03 | 1.06 | 3.13E−06 |
| 16:53842908 | rs12149832 | FTO | intron | A/G | 0.41 | 1.07 | 1.06 | 1.09 | 9.05E−20 |
| 16:69566309 | rs12933292 | NFAT5* | intergenic | C/G | 0.59 | 1.04 | 1.03 | 1.06 | 2.75E−07 |
| 17:2097583 | rs1002135 | SMG6* | intron | G/T | 0.38 | 1.04 | 1.02 | 1.06 | 5.89E−07 |
| 17:37834715 | rs12150603 | PNMT* | intron | G/A | 0.35 | 1.05 | 1.03 | 1.06 | 5.78E−08 |
| 17:57486425 | rs150947345 | YPEL2* | flanking | A/T | 0.02 | 1.16 | 1.09 | 1.23 | 1.62E−06 |
| 17:65880259 | rs34432450 | BPTF* | intron | C/T | 0.21 | 1.06 | 1.04 | 1.08 | 3.30E−10 |
| 18:36560942 | rs79329549 | 18g12.2* | intergenic | T/G | 0.91 | 1.08 | 1.05 | 1.11 | 5.24E−08 |
| 21:30534128 | rs1999323 | MAP3K7CL* | intron | T/C | 0.15 | 1.05 | 1.03 | 1.07 | 4.04E−06 |

TABLE 7B
HF in MVP EUR, additionally adjusted for BMI.

| | | | | | | HF in MVP EUR, | | | |
|---|---|---|---|---|---|---|---|---|---|
| | | | | Risk | | additionally adjusted for BMI | | | |
| | | | | allele | Risk | | Lower | Upper | |
| | | Closest | Genomic | Ref | allele | | 95% | 95% | p- |
| Position | rsID. | Gene | Region | allele | freq. | OR | CI | CI | value |
| 1:16310737 | rs371236917 | SRARP/ | flanking | C/CT | 0.7 | 1.06 | 1.04 | 1.07 | 6.43E−12 |
| | | HSPB7/ | | | | | | | |
| | | ZBTB17 | | | | | | | |
| 1:109822143 | †rs1277930 | CELSR2 | flanking | A/G | 0.77 | 1.05 | 1.03 | 1.06 | 3.08E−07 |
| 2:11568158 | †rs7595697 | E2F6* | flanking | T/C | 0.37 | 1.04 | 1.02 | 1.06 | 4.02E−07 |
| 3:44005735 | †rs6795366 | ABHD5* | intergenic | C/T | 0.74 | 1.03 | 1.02 | 1.05 | 8.34E−05 |
| 4:111665783 | rs2634073 | PITX2 | intergenic | T/C | 0.2 | 1.07 | 1.05 | 1.09 | 4.54E−11 |
| 6:36647289 | rs3176326 | CDKN1A | intron | G/A | 0.8 | 1.08 | 1.06 | 1.1 | 4.60E−17 |
| 6:161010118 | rs10455872 | LPA | intron | G/A | 0.07 | 1.12 | 1.09 | 1.15 | 9.68E−15 |
| 7:74068167 | rs4717903 | GTF2I* | flanking | C/T | 0.25 | 1.03 | 1.02 | 1.05 | 2.17E−04 |
| 9:22124744 | †rs4977575 | CDKN2B-AS | intergenic | G/C | 0.49 | 1.06 | 1.05 | 1.08 | 6.75E−18 |
| 9:136154168 | rs579459 | ABO | flanking | C/T | 0.22 | 1.05 | 1.03 | 1.06 | 1.25E−06 |
| 10:75583034 | rs59693993 | CAMK2G | intron | C/T | 0.86 | 1.05 | 1.03 | 1.07 | 1.12E−05 |
| 10:121422836 | rs61869036 | BAG3 | intron | G/C | 0.79 | 1.04 | 1.03 | 1.06 | 1.42E−06 |
| 16:53842908 | rs12149832 | FTO | intron | A/G | 0.41 | 1.04 | 1.02 | 1.05 | 2.23E−06 |
| 16:69566309 | rs12933292 | NFAT5* | intergenic | C/G | 0.59 | 1.03 | 1.02 | 1.05 | 4.04E−05 |
| 17:2097583 | rs1002135 | SMG6* | intron | G/T | 0.38 | 1.04 | 1.02 | 1.05 | 5.93E−06 |
| 17:37834715 | rs12150603 | PNMT* | intron | G/A | 0.35 | 1.05 | 1.03 | 1.06 | 8.33E−08 |
| 17:57486425 | rs150947345 | YPEL2* | flanking | A/T | 0.02 | 1.15 | 1.08 | 1.22 | 6.42E−06 |
| 17:65880259 | rs34432450 | BPTF* | intron | C/T | 0.21 | 1.06 | 1.04 | 1.07 | 1.37E−08 |
| 18:36560942 | rs79329549 | 18g12.2* | intergenic | T/G | 0.91 | 1.06 | 1.04 | 1.09 | 2.12E−06 |
| 21:30534128 | rs1999323 | MAP3K7CL* | intron | T/C | 0.15 | 1.05 | 1.03 | 1.08 | 1.81E−06 |

TABLE 7C
HF in MVP EUR, additionally adjusted for diabetes.

| | | | | | | HF in MVP EUR, | | | |
|---|---|---|---|---|---|---|---|---|---|
| | | | | Risk | | additionally adjusted for BMI | | | |
| | | | | allele | Risk | | Lower | Upper | |
| | | Closest | Genomic | Ref | allele | | 95% | 95% | p- |
| Position | rsID. | Gene | Region | allele | freq. | OR | CI | CI | value |
| 1:16310737 | rs371236917 | SRARP/ | flanking | C/CT | 0.7 | 1.06 | 1.04 | 1.07 | 4.08E−12 |
| | | HSPB7/ | | | | | | | |
| | | ZBTB17 | | | | | | | |
| 1:109822143 | †rs1277930 | CELSR2 | flanking | A/G | 0.77 | 1.05 | 1.03 | 1.07 | 5.77E−08 |
| 2:11568158 | †rs7595697 | E2F6* | flanking | T/C | 0.37 | 1.04 | 1.02 | 1.05 | 1.02E−06 |
| 3:44005735 | †rs6795366 | ABHD5* | intergenic | C/T | 0.74 | 1.04 | 1.02 | 1.06 | 7.00E−06 |
| 4:111665783 | rs2634073 | PITX2 | intergenic | T/C | 0.2 | 1.07 | 1.05 | 1.09 | 1.48E−11 |
| 6:36647289 | rs3176326 | CDKN1A | intron | G/A | 0.8 | 1.07 | 1.06 | 1.09 | 1.41E−15 |
| 6:161010118 | rs10455872 | LPA | intron | G/A | 0.07 | 1.11 | 1.08 | 1.14 | 6.94E−13 |
| 7:74068167 | rs4717903 | GTF2I* | flanking | C/T | 0.25 | 1.04 | 1.02 | 1.06 | 1.89E−05 |
| 9:22124744 | †rs4977575 | CDKN2B-AS | intergenic | G/C | 0.49 | 1.06 | 1.04 | 1.08 | 8.38E−16 |
| 9:136154168 | rs579459 | ABO | flanking | C/T | 0.22 | 1.04 | 1.02 | 1.06 | 1.81E−05 |
| 10:75583034 | rs59693993 | CAMK2G | intron | C/T | 0.86 | 1.05 | 1.03 | 1.07 | 1.67E−06 |
| 10:121422836 | rs61869036 | BAG3 | intron | G/C | 0.79 | 1.04 | 1.02 | 1.06 | 3.74E−06 |
| 16:53842908 | rs12149832 | FTO | intron | A/G | 0.41 | 1.07 | 1.05 | 1.08 | 2.23E−16 |
| 16:69566309 | rs12933292 | NFAT5* | intergenic | C/G | 0.59 | 1.04 | 1.02 | 1.05 | 1.95E−06 |
| 17:2097583 | rs1002135 | SMG6* | intron | G/T | 0.38 | 1.04 | 1.02 | 1.05 | 6.04E−07 |
| 17:37834715 | rs12150603 | PNMT* | intron | G/A | 0.35 | 1.04 | 1.03 | 1.06 | 1.34E−07 |
| 17:57486425 | rs150947345 | YPEL2* | flanking | A/T | 0.02 | 1.16 | 1.1 | 1.22 | 2.82E−06 |
| 17:65880259 | rs34432450 | BPTF* | intron | C/T | 0.21 | 1.06 | 1.04 | 1.08 | 2.12E−09 |
| 18:36560942 | rs79329549 | 18q12.2* | intergenic | T/G | 0.91 | 1.07 | 1.04 | 1.1 | 1.57E−07 |
| 21:30534128 | rs1999323 | MAP3K7CL* | intron | T/C | 0.15 | 1.05 | 1.03 | 1.07 | 3.37E−06 |

The bold indicates that additional covariate adjustment reduced the genetic association with HF. Chromosomal position is based on GCh37/hg19 reference. The sentinel SNPs were mapped to the closed refseq genes based on chromosomal base-pair position. The genetic associations were aligned to effects of the risk alleles (i.e., increased risk for HF). OR: odds ratio; CI: confidence interval; MVP—Million Veteran Program cohort; EUR: European ancestry.

Tables 8-C. Sensitivity analyses of genome-wide significant sentinel variants associated with HFrEF or HFpEF additionally adjusted for BMI or diabetes status.

TABLE 8A
HF in MVP EUR.

| | | | | Risk | | HF in MVP EUR | | | |
|---|---|---|---|---|---|---|---|---|---|
| | | | | allele | Risk | | Lower | Upper | |
| | | Closest | Genomic | Ref | allele | | 95% | 95% | p- |
| Position | rsID. | Gene | Region | allele | freq. | OR | CI | CI | value |
| HFrEF | | | | | | | | | |
| 1:16335527 | rs1763610 | HSPB7 | flanking | C/G | 0.64 | 1.11 | 1.08 | 1.13 | 1.06E−18 |
| 1:61881191 | rs2261792 | NFIA | intron | G/A | 0.36 | 1.06 | 1.04 | 1.09 | 4.11E−08 |
| 2:11568740 | rs 12612948 | E2F6 | flanking | G/A | 0.35 | 1.07 | 1.04 | 1.09 | 1.27E−08 |
| 3:69824230 | rs56286049 | MITF | intron | C/G | 0.77 | 1.08 | 1.05 | 1.11 | 7.86E−09 |
| 6:12903957 | rs9349379 | PHACTR1 | intron | G/A | 0.4 | 1.06 | 1.04 | 1.09 | 5.53E−09 |
| 6:36645988 | rs4151702 | CDKN1A | intron | G/C | 0.79 | 1.15 | 1.12 | 1.18 | 7.29E−25 |
| 6:161010118 | rs10455872 | LPA | intron | G/A | 0.07 | 1.14 | 1.1 | 1.19 | 2.17E−11 |
| 9:22124744 | rs4977575 | CDKN2B- | intergenic | G/C | 0.49 | 1.08 | 1.06 | 1.11 | 1.80E−13 |
| | | AS | | | | | | | |
| 10:121429633 | rs2234962 | BAG3 | missense | T/C | 0.79 | 1.12 | 1.09 | 1.15 | 9.02E−18 |
| 12:51320290 | rs7306330 | METTL7A | intron | A/T | 0.42 | 1.07 | 1.05 | 1.09 | 5.58E−10 |
| 16:53834607 | rs7188250 | FTO | intron | C/T | 0.41 | 1.07 | 1.04 | 1.09 | 2.85E−09 |
| 17:37824339 | rs3764351 | PNMT | intron | G/A | 0.36 | 1.07 | 1.05 | 1.09 | 4.34E−09 |
| 17:65852907 | rs4790908 | BPTF | intron | G/T | 0.2 | 1.08 | 1.05 | 1.11 | 3.04E−09 |
| HFpEF | | | | | | | | | |
| 16:53802494 | rs11642015 | FTO | intron | T/C | 0.4 | 1.07 | 1.05 | 1.1 | 6.45E−11 |

TABLE 8B
HF in MVP EUR, additionally adjusted for BMI.

| | | | | | | HF in MVP EUR, | | | |
|---|---|---|---|---|---|---|---|---|---|
| | | | | Risk | | additionally adjusted for BMI | | | |
| | | | | allele | Risk | | Lower | Upper | |
| | | Closest | Genomic | Ref | allele | | 95% | 95% | p- |
| Position | rsID. | Gene | Region | allele | freq. | OR | CI | CI | value |
| HFrEF | | | | | | | | | |
| 1:16335527 | rs1763610 | HSPB7 | flanking | C/G | 0.64 | 1.1 | 1.08 | 1.12 | 2.34E−18 |
| 1:61881191 | rs2261792 | NFIA | intron | G/A | 0.36 | 1.06 | 1.04 | 1.09 | 5.50E−08 |
| 2:11568740 | rs12612948 | E2F6 | flanking | G/A | 0.35 | 1.07 | 1.04 | 1.09 | 8.94E−09 |
| 3:69824230 | rs56286049 | MITF | intron | C/G | 0.77 | 1.08 | 1.05 | 1.1 | 1.74E−09 |
| 6:12903957 | rs9349379 | PHACTR1 | intron | G/A | 0.4 | 1.06 | 1.04 | 1.09 | 1.16E−08 |
| 6:36645988 | rs4151702 | CDKN1A | intron | G/C | 0.79 | 1.14 | 1.11 | 1.16 | 5.11E−26 |
| 6:161010118 | rs10455872 | LPA | intron | G/A | 0.07 | 1.15 | 1.11 | 1.2 | 4.33E−12 |
| 9:22124744 | rs4977575 | CDKN2B- | intergenic | G/C | 0.49 | 1.08 | 1.06 | 1.1 | 2.86E−14 |
| | | AS | | | | | | | |
| 10:121429633 | rs2234962 | BAG3 | missense | T/C | 0.79 | 1.11 | 1.09 | 1.13 | 9.33E−18 |
| 12:51320290 | rs7306330 | METTL7A | intron | A/T | 0.42 | 1.07 | 1.05 | 1.1 | 3.66E−10 |
| 16:53834607 | rs7188250 | FTO | intron | C/T | 0.41 | 1.04 | 1.02 | 1.07 | 5.72E−05 |
| 17:37824339 | rs3764351 | PNMT | intron | G/A | 0.36 | 1.07 | 1.04 | 1.09 | 8.30E−09 |
| 17:65852907 | rs4790908 | BPTF | intron | G/T | 0.2 | 1.08 | 1.05 | 1.11 | 2.17E−08 |
| HFpEF | | | | | | | | | |
| 16:53802494 | rs11642015 | FTO | intron | T/C | 0.4 | 1.02 | 1 | 1.04 | 4.48E−02 |

TABLE 8C
HF in MVP EUR, additionally adjusted for diabetes.

| | | | | | | HF in MVP EUR, | | | |
|---|---|---|---|---|---|---|---|---|---|
| | | | | | | additionally adjusted for | | | |
| | | | | Risk | | diabetes | | | |
| | | | | allele | Risk | | Lower | Upper | |
| | | Closest | Genomic | Ref | allele | | 95% | 95% | p- |
| Position | rsID. | Gene | Region | allele | freq. | OR | CI | CI | value |
| HFrEF | | | | | | | | | |
| 1:16335527 | rs1763610 | HSPB7 | flanking | C/G | 0.64 | 1.1 | 1.08 | 1.12 | 2.16E−18 |
| 1:61881191 | rs2261792 | NFIA | intron | G/A | 0.36 | 1.06 | 1.04 | 1.09 | 3.88E−08 |
| 2:11568740 | rs12612948 | E2F6 | flanking | G/A | 0.35 | 1.07 | 1.04 | 1.09 | 1.05E−08 |
| 3:69824230 | rs56286049 | MITF | intron | C/G | 0.77 | 1.07 | 1.05 | 1.1 | 1.21E−08 |
| 6:12903957 | rs9349379 | PHACTR1 | intron | G/A | 0.4 | 1.07 | 1.04 | 1.09 | 2.96E−09 |
| 6:36645988 | rs4151702 | CDKN1A | intron | G/C | 0.79 | 1.13 | 1.11 | 1.16 | 1.16E−24 |
| 6:161010118 | rs10455872 | LPA | intron | G/A | 0.07 | 1.15 | 1.1 | 1.19 | 1.64E−11 |
| 9:22124744 | rs4977575 | CDKN2B- | intergenic | G/C | 0.49 | 1.08 | 1.06 | 1.1 | 3.38E−13 |
| | | AS | | | | | | | |
| 10:121429633 | rs2234962 | BAG3 | missense | T/C | 0.79 | 1.11 | 1.08 | 1.13 | 1.59E−17 |
| 12:51320290 | rs7306330 | METTL7A | intron | A/T | 0.42 | 1.07 | 1.05 | 1.1 | 3.71E−10 |
| 16:53834607 | rs7188250 | FTO | intron | C/T | 0.41 | 1.06 | 1.04 | 1.08 | 8.81E−08 |
| 17:37824339 | rs3764351 | PNMT | intron | G/A | 0.36 | 1.07 | 1.04 | 1.09 | 9.11E−09 |
| 17:65852907 | rs4790908 | BPTF | intron | G/T | 0.2 | 1.08 | 1.05 | 1.11 | 1.12E−08 |
| HFpEF | | | | | | | | | |
| 16:53802494 | rs11642015 | FTO | intron | T/C | 0.4 | 1.07 | 1.04 | 1.09 | 5.02E−09 |

The bold indicates that additional covariate adjustment reduced the genetic association with HFrEF or HFpEF. Chromosomal position is based on GCh37/hg19 reference. The sentinel SNPs were mapped to the closed refseq genes based on chromosomal base-pair position. The genetic associations were aligned to effects of the risk alleles (i.e., increased risk for HF subtypes). OR: odds ratio; CL: confidence interval; MVP—Million Veteran Program cohort; EUR: European ancestry.

Genetic Correlation between HFrEF/HFpEF and HF-Related Traits. Analysis of shared genetic contributions between HF subtypes and HF-related traits demonstrated significant genetic correlation between each HF subtype and risk factors (FIG. 14). Similar positive correlations were observed for HFrEF and HFpEF with CAD, AF, triglycerides, diastolic blood pressure and a similar negative correlation with HDL-C. HFpEF had higher correlation than HFrEF in T2D3 (rHFpEF=0.54±0.05, rHFpEF=0.42±0.04), BMI (rHFpEF=0.65±0.04, rHFpEF=0.41±0.03), systolic blood pressure (rHFpEF=0.42±0.05, rHFpEF=0.29±0.04) and pulse pressure (rHFpEF=0.36±0.05, rHFpEF=0.16±0.04). The genetic correlations between BMI, T2D3 and HFpEF are comparable to that between HFpEF and HFrEF (0.57±0.07). A significant correlation of HFrEF or HFpEF with LDL or total cholesterol, or eGFR was not observed. Using LDSC and the MVP GWAS summary statistics, we estimated the heritability (h2) of composite HF, HFpEF and HFrEF as 3.7% (SE 0.3%), 1.9% (SE 0.2%) and 3.1% (0.3%), respectively. Heritability of HFpEF was substantially lower than that of composite HF and HFrEF.

Mendelian Randomization Association Analysis of HF Risk Factors. The MR association results from the inverse-variance-weighted method was used since the assumption of zero-intercept was not violated in the Egger regression. In primary MR analyses (inverse-variance-weighted estimates), CAD had a stronger causal association with HFrEF, and the lipid parameters as well as T2D and DBP had a significant causal association with HFrEF. While AF, BMI, and SBP demonstrated similar causal associations with both HF subtypes, PP was significantly associated with HFpEF. Similar results were observed from the median weighted method. Sensitivity analysis using Egger regression showed consistent effect estimates but larger confidence intervals.

Discussion. The study demonstrated significant differences in the genetic architecture of HFrEF and HFpEF thereby indicating clear differences in the pathobiology of HFrEF and HFpEF. Genetic associations at the level of individual variants were different between the two subtypes of HF, as were the magnitude of genetic correlations between each subtype of HF and various known risk factors for HF. Using Mendelian Randomization, it was found that risk factors influence HFrEF and HFpEF differently. Creating a sub-cohort of HFrEF increased genetic discovery in spite of reducing sample size to approximately half that of unclassified HF. Conversely, genetic discovery for HFpEF remained limited despite a robust number of cases similar in number to HFrEF. The findings in HFpEF clearly indicate the clinical heterogeneity of HFpEF and the need for further sub-phenotyping of HFpEF to uncover specific pathobiology and therapeutic options.

Genetic analyses was used to examine associations of HF subtypes and their risk factors, and the differential causal contributions of HF risk factors to HFrEF and HFpEF. Coronary artery disease was more genetically correlated and causally related with HFrEF than with HFpEF. Lipid markers that are associated with CAD showed causal associations with HFrEF. Pulse pressure was more correlated and causally associated with HFpEF; this finding may be related to the importance of aortic stiffness and ventriculo-aortic coupling in the pathogenesis of HFpEF. Atrial fibrillation was causally related to both HFrEF and HFpEF, while type 2 diabetes mellitus demonstrated a stronger causal association with HFrEF. BMI was strongly associated with both HFpEF and HFrEF; FTO, which is strongly associated with BMI, was the locus significantly associated with HF, HFrEF and HFpEF. While there were some variations between the results of the genetic correlation analyses which measured shared heritability between HF risk factor and specific HF sub-type and the Mendelian randomization analyses which examined causal relations of HF risk factors to each HF sub-type, the overall results were similar between the two methods. For example, the associations of CAD and the lipid traits were more strongly associated with HFrEF, even though there were differences in the degree of associations with HFrEF and HFpEF when comparing the results of genetic correlation and Mendelian Randomization. The genetic correlation between HFrEF and HFpEF was modest (r2 approximately 32%) and in the range of association of individual risk factors with each subtype of HF, reinforcing the findings at the genomic level that HFrEF and HFpEF have different genetic architecture.

The PNMT gene encodes phenylethanolamine N-methyltransferase, which catalyzes the conversion of epinephrine to norepinephrine. Inappropriate sympathetic activation and elevated catecholamine levels is a major pathophysiologic substrate and therapeutic target in HFrEF. Polymorphisms of the PNMT gene are associated with resting and post-exercise catecholamines and with increased risk of hypertension (Huang C, et al. Am J Hypertens. 2011; 24:1222-6). The gene E2F6 codes for a member of the E2F family of transcription factors that regulate cardiac development, cardiomyocyte growth, and myocardial metabolism; deletion of this gene leads to early onset cardiomyopathy (Major J L, et al. PLoS One. 2017; 12:e0170066). A cardiac-specific isoform of microphthalmia transcription factor (MITF) regulates the hypertrophic response of the myocardium (Tshori S, et al. J Clin Invest. 2006; 116:2673-81). The transcription factor NFIA, which has major roles in glial cell development, has been associated with QRS duration by a GWAS (Evans D S, et al. Eur J Heart Fail. 2020; 22:54-66). While the function of Methyltransferase Like 7A (METTL7A) is not well understood, other methyltranferases such as METTL3 and −14 methylate N6-adenosine moieties in RNA and oppose the action of FTO, a N6-adenosine demethylase, which is the gene that was significantly associated with HF, HFrEF, and HFpEF; myocardial changes in N6-adenosine methylation of mRNA is associated with progression to HF (Evans D S, et al. Eur J Heart Fail. 2020; 22:54-66).

The lack of other large adequately phenotyped cohorts of HFpEF and HFrEF prevented external replication in the HF subtypes. The cohort used for this study was different from a typical population-based cohort in that it was composed predominantly of older males, and since participants were recruited in hospital settings, a higher prevalence of heart failure and a higher prevalence of comorbidities in the control population was observed. While cases and controls were not matched, the fact that there was a high prevalence of comorbidities in the control population may have limited the significant associations to variants that are truly associated with heart failure and not with comorbidities per se. While the cohort used in this study had fewer women with HFpEF compared to the general population, genetic associations with risk factors in the predominantly older male cohort of HFpEF, such as less association with CAD compared to HFrEF, were similar to reports in other epidemiologic cohorts. A high prevalence of HF and of comorbidities in the genotyped cohort was observed; this is likely because MVP uses a hospital-based recruitment process. The high prevalence of comorbidities in the control population may have decreased the number of significantly associated loci; however, the relative matching of the control population may have increased the likelihood that the discovered loci were more likely to be associated with the pathobiology of HF independent of their effects on HF risk factors.

The findings in the HFpEF cohort were analyzed to confirm that the lack of novel genetic discovery was not due to issues of curation of the phenotype from the EHR. The current universal definition of HFpEF17 was used, and natural language processing was also utilized to obtain the LVEF closest to date of diagnosis. In addition, to ensure that the findings reported herein were not due to issues in curating the HFpEF phenotype from the EHR, the more restrictive phenotype was used based on measurement of natriuretic peptides and use of diuretics (positive predictive value of 96%) (Patel Y R, et al. BMC Cardiovasc Disord. 2018; 18:128) and GWAS was repeated in this more restrictively curated sub-group of HFpEF, and similar genetic associations were found but less statistical power (due to smaller sample size) comparing to the main HFpEF cohort. The HFpEF cohort was similar in clinical profile to the HFrEF cohort, and had similar clinical characteristics and comorbidity burden as the HFpEF cohorts described in other epidemiological studies (except for the lack of inclusion of other ethnic minorities in the current study). The HFpEF cohort was also similar to HFpEF cohorts in major clinical trials of HFpEF in spite of varying inclusion/exclusion criteria and higher enrollment of women and minorities in those clinical trials (Solomon S D, et al. N Engl J Med. 2019; 381:1609-1620). More specifically, comparison of top HFpEF-associated between GWAS results from two HFpEF definitions—a more restrictive definition (n=12,119 cases) and the definition used for the primary HFpEF GWAS (n=19,598 cases). Similar to the primary HFpEF GWAS, one genome-wide significant locus (FTO) was identified. Using LDSC and the GWAS summary statistics, it was found that the genetic correlation between the two HFpEF definitions was very high (r=0.981, p<2×10⁻¹⁶). Among top 110 HFpEF-associated common SNPs (p<10−6, MAF>1%), the genetic effects between the two HFpEF GWAS were highly correlated (r=0.995, p<2×10⁻¹⁶). Mostly driven by a larger number of HFpEF cases in the original definition (19,598 vs. 12,119), the p-values of 109 out of 110 SNPs were lower in the original HFpEF GWAS conducted in the less restrictive cohort. Hence, the results described herein demonstrate that HFpEF is a clinically heterogenous condition, and that sub-phenotyping can advance the biologic and therapeutic understanding of this condition. To tackle the heterogeneity, both a priori grouping into sub-phenotypes (Shah S J, et al. Circulation. 2016; 134:73-90), and unbiased clustering using machine learning approaches (Uijl A, et al. Identification of distinct phenotypic clusters in heart failure with preserved ejection fraction. Eur J Heart Fail. 2021) have both been used.

In conclusion, these results demonstrate a striking difference in the genetic underpinnings of HFrEF and HFpEF and support the urgent need for novel approaches to sub-phenotype HFpEF to enable prevention and treatment. Better genetic understanding of HF subtypes can be used to precisely diagnosis, accurately assess risk, and effectively treat and management the global pandemic of heart failure.

What is claimed is:

1. A method of identifying a subject at risk for developing heart failure (HF), heart failure with preserved ejection fraction (HFpEF), or heart failure with reduced ejection fraction (HFrEF), the method comprising:
a) obtaining a biological sample from the subject or having obtained a biological sample from the subject;
b) determining the presence of one or more variants of one or more of E2F6, MITF, NFIA, METTL7A, FTO and PNMT in the biological sample; and
c) identifying the subject at risk for developing HF when the one or more variants of the one or more of E2F6, MITF, NFIA, and METTL7A is present in the biological sample;
identifying the subject at risk for developing HFrEF when the one or more variants of PMNT is present in the biological sample; or
identifying the subject at risk for developing HFpEF, when the one or more variants of FTO is present in the biological sample.

2. A method of identifying heart failure in a subject that is responsive to treatment with a beta blocker, the method comprising:
a) obtaining a biological sample from the subject or having obtained a biological sample from the subject;
b) determining the presence of one or more variants of PNMT in the biological sample of step a);
c) contacting the biological sample in step b) with the beta blocker;
d) determining a change in expression levels of PNMT in the biological sample of step c);
e) identifying the heart failure in the subject as responsive to the beta blocker when the level of expression of PNMT is different than the level of expression of PNMT in step b).

3. A method of treating heart failure (HF), heart failure with preserved ejection fraction (HFpEF), or heart failure with reduced ejection fraction (HFrEF) in a subject, the method comprising:
a) obtaining a biological sample from the subject or having obtained a biological sample from the subject;
b) determining the presence of one or more variants of one or more of E2F6, MITF, NFIA, METTL7A, FTO and PNMT in the biological sample;
c) identifying the subject having HF when the one or more variants of the one or more of E2F6, MITF, NFIA, and METTL7A is present in the biological sample; identifying the subject as having HFrEF when the one or more variants of PNMT is present in the biological sample; or identifying the subject as having HFpEF, when the one or more variants of FTO is present in the biological sample; and
d) administering to the subject in step c) a therapeutically effect amount of a beta blocker, a regimen of electrocardiograms or a combination thereof, thereby treating heart failure (HF), heart failure with preserved ejection fraction (HFpEF), or heart failure with reduced ejection fraction (HFrEF) in the subject.

4. A method of treating a heart failure with reduced ejection fraction (HFrEF) in a subject who is responsive to a beta blocker, wherein the method comprises the steps of:
a) selecting a subject with heart failure with reduced ejection fraction (HFrEF) who is responsive to treatment with a beta blocker by:
i) obtaining a biological sample from the subject or having obtained a biological sample from the subject;
ii) determining the presence of one or more variants of PNMT in the biological sample of step i); and
b) based on the presence of one or more variants of PNMT, treating the heart failure patient with the beta blocker, thereby treating heart failure with reduced ejection fraction (HFrEF) in the subject.

5. The method of claim 4, further comprising: iii) contacting the biological sample in step ii) with the beta blocker; iv) determining a change in expression levels of PNMT in the biological sample of step iii); and v) identifying the heart failure with reduced ejection fraction (HFrEF) in the patient as responsive to the beta blocker when the level of expression of PNMT is different than the level of expression of PNMT in step ii) after step a) ii).

6. The method of any of the preceding claims, wherein the one or more variants of E2F6 is a T to C at position 2:11568158 or a G to A at position 2:11568740.

7. The method of any of the preceding claims, wherein the one or more variants of one or more of MITF is a C to G a position 3:69824230.

8. The method of any of the preceding claims, wherein the one or more variants of one or more of NFIA is a G to A at position 1:61881191.

9. The method of any of the preceding claims, wherein the one or more variants of one or more of METTL7A is an A to T at position 12:51320290.

10. The method of any of the preceding claims, wherein the one or more variants of one or more of FTO is a T to C at position 16:53802494.

11. The method of any of the preceding claims, wherein the one or more variants of one or more of PNMT is a G to A at position 17:37824339.

12. The method of any of the preceding claims, wherein the biological sample is a blood sample or a nucleic acid sample.

13. The method of any of the preceding claims, wherein the beta blocker is metoprolol succinate, bisoprolol, or carvedilol.

14. The method of any of the preceding claims, wherein the step the presence of one or more variants of E2F6, MITF, NFIA, METTL7A, FTO and PNMT is determined using quantitative PCR, RNA-sequencing, next generation sequencing or a combination thereof.

15. A method of determining whether a subject with heart failure (HF), heart failure with preserved ejection fraction (HFpEF), or heart failure with reduced ejection fraction (HFrEF) will respond to a therapeutic treatment, the method comprising:
a) determining the presence of one or more variants of at least one biomarker selected from the group consisting of E2F6, MITF, NFIA, METTL7A, FTO and PNMT in a sample obtained from the subject before the treatment; and
b) determining a change in the expression level measured at step a) before and after contacting the sample with the therapeutic treatment;
wherein detecting a difference in the biomarker expression level between the sample before and after contact with the therapeutic treatment is indicative that the subject will respond to the therapeutic treatment.

16. A gene expression panel assessing risk of developing heart failure (HF), heart failure with preserved ejection fraction (HFpEF), or heart failure with reduced ejection fraction (HFrEF) in a human subject, consisting of primers or probes for detecting one or more of E2F6, MITF, NFIA, METTL7A, FTO and PNMT in a sample.
